(19) United States
(12) Patent Application Publication
(10) Pub. No.: US 2005/0222448 A1
(43) Pub. Date: Oct. 6, 2005

(54) N-sulphonylaminoacetonitriles having pesticidal properties

(76) Inventors: Arthur Steiger, Arlesheim (CH); Werner Zambach, Battwil (CH); Jacques Bouvier, Neuchatel (CH); Pierre Ducray, Village-Neuf (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/518,063

(22) PCT Filed: Jun. 18, 2003
(86) PCT No.: PCT/EP03/06483

§ 371 Date: Jan. 18, 2005

(30) Foreign Application Priority Data
Jun. 19, 2002 (GB) 0214117.4

(51) Int. Cl. A61K 31/275

(52) U.S. Cl. 558/390; 514/522

(57) ABSTRACT

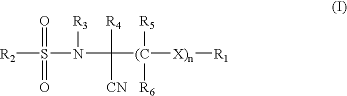

The invention relates to compounds of the general formula (I) and, if appropriate, enantiomers thereof. The active compounds have advantageous pesticidal properties. They are suitable, in particular, for the control of parasites in warm-blooded animals.

The present invention relates to novel sulphonylaminoacetonitrile compounds of the formula 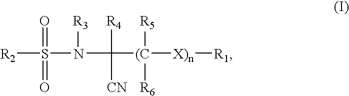
in which
R₁ is aryl or heteroaryl, in each case unsubstituted or mono- or polysubstituted by R₇, where the substituents can in each case be identical or different if their number is greater than 1;
R₂ is C₁-C₆alkyl, halo-C₁-C₆alkyl, C₃-C₈cycloalkyl, halo-C₃-C₈cycloalkyl, NHR₈; aryl or heteroaryl, in each case unsubstituted or mono- or polysubstituted by R₇, where the substituents can in each case be identical or different if their number is greater than 1, or pyrrolidinyl, piperidinyl, imidazolidinyl, piperazinyl, pyrazolidinyl, morpholinyl, indolinyl or isoindolinyl, in each case bonded via N;
R₃ is hydrogen, C₁-C₆alkyl, halo-C₁-C₆alkyl, C₁-C₆alkoxy-C₁-C₆alkyl, benzyl, C₁-C₆alkylheteroaryl, C₁-C₆alkoxycarbonyl or C₁-C₆alkylcarbonyl;
R₄, R₅ and R₆ either independently of one another are hydrogen, halogen, C₁-C₆alkyl, halo-C₁-C₆alkyl, C₁-C₆alkoxy, halo-C₁-C₆alkoxy, C₁-C₆alkylthio, halo-C₁-C₆alkylthio, C₂-C₆alkenyl, C₂-C₆alkynyl, unsubstituted or substituted C₃-C₈cycloalkyl, where the substituents are selected from the group consisting of halogen and C₁-C₆alkyl, or unsubstituted or substituted phenyl, where the substituents are selected from the group consisting of halogen, C₁-C₆alkyl and phenyl;
or R₄ and R₅, together with the carbon atoms to which they are bonded, are a five- to seven-membered, saturated or partially unsaturated heterocyclic ring having 1 or 2 heteroatoms from the group consisting of nitrogen, oxygen and sulphur;
R₇ is halogen, C₁-C₆alkyl, halo-C₁-C₆alkyl, C₁-C₆alkoxy, halo-C₁-C₆alkoxy, C₁-C₆alkylthio, halo-C₁-C₆alkylthio, C₂-C₆alkenyl, C₂-C₆alkynyl; aryl, phenylacetylenyl or heteroaryl, in each case unsubstituted or mono- or polysubstituted, where the substituents are in each case selected from the group consisting of halogen, nitro, cyano, C₁-C₆alkyl, halo-C₁-C₆alkyl, C₁-C₆alkoxy, halo-C₁-C₆alkoxy, and can in each case be identical or different if their number is greater than 1;
R₈ is aryl which is unsubstituted or mono- to pentasubstituted, where the substituents are selected from the group consisting of halogen, nitro, cyano, C₁-C₆alkyl, halo-C₁-C₆alkyl, C₁-C₆alkoxy and halo-C₁-C₆alkoxy, and can be identical or different if their number is greater than 1;
X is O, S, S(O) or S(O)₂; and
n is 0 or 1;
and, if appropriate, E/Z isomers, E/Z isomer mixtures and/or tautomers, in each case in free form or in salt form;
a process for preparation and the use of these compounds, their isomers and tautomers; starting compounds for the preparation of the compounds of the formula (I); pesticides whose active compound is selected from the compounds of the formula (I) and their tautomers; and a process for the control of plant-injurious insects and representatives of the order Acarina, and of endo- and ectoparasites, in particular helminths, in and on warm-blooded agricultural and domestic animals, using these compositions.

Substituted aminoacetonitrile compounds having pesticidal action are described in the literature. The active compounds actually disclosed there however, cannot always fulfil the requirements with respect to potency and spectrum of action. There is thus a need for active compounds having improved pesticidal properties. It has now been found that the compounds of the formula (I) according to the invention have outstanding pesticidal properties, in particular against endo- and ectoparasites in and on agricultural and domestic animals and plants.

Some compounds of formula (I) may be in the form of tautomers. The compounds of formula (I) are therefore to be understood hereinbefore and hereinafter as including corresponding tautomers, where appropriate, even if the latter are not specifically mentioned in each case.

The compounds of formula (I) and, where appropriate, tautomers thereof are capable of forming salts, for example acid addition salts. Those acid addition salts are formed, for example, with strong inorganic acids, such as mineral acids, e.g. sulfuric acid, a phosphoric acid or a hydrohalic acid, with strong organic carboxylic acids, such as unsubstituted or substituted, e.g. halo-substituted, C₁-C₄alkanecarboxylic acids, e.g. acetic acid, saturated or unsaturated dicarboxylic acids, e.g. oxalic, malonic, maleic, fumaric and phthalic acid, hydroxycarboxylic acids, e.g. ascorbic, lactic, malic, tartaric and citric acid, or benzoic acid, or with organic sulfonic acids, such as unsubstituted or substituted, e.g. halo-substituted, C₁-C₄alkane- or aryl-sulfonic acids, e.g. methane- or p-toluene-sulfonic acid. Furthermore, compounds of formula (I) having at least one acid group are capable of forming salts with bases. Suitable salts with bases are, for example, metal salts, such as alkali metal and alkaline earth metal salts, e.g. sodium, potassium and magnesium salts, and salts with ammonia or an organic amine, such as morpholine, piperidine, pyrrolidine, a mono-, di- or tri-lower alkylamine, e.g. ethyl-, diethyl-, triethyl- or dimethyl-propyl-amine, or a mono-, di- or tri-hydroxy-lower alkylamine, e.g. mono-, di- or tri-ethanolamine. In addition, corresponding internal salts may optionally also be formed. Preference is given, firstly, to the free form. Among the salts of compounds of formula (I) preference is given to agrochemically advantageous salts. The free compounds of formula (I) and salts thereof are to be understood hereinbefore and hereinafter as including, where appropriate, both the corresponding salts and the free compounds of formula (I), respectively. The same is correspondingly true for tautomers of compounds of formula (I) and salts thereof.

The general terms used hereinbefore and hereinafter have the following meanings, unless defined otherwise.

Aryl is phenyl or naphthyl.

Heteroaryl is pyridyl, pyrimidyl, s-triazinyl, 1,2,4-triazinyl, thienyl, furanyl, pyrryl, pyrazolyl, imidazolyl, thiazolyl, triazolyl, oxazolyl, thiadiazolyl, oxadiazolyl, benzothienyl, benzofuranyl, benzothiazolyl, indolyl or indazolyl, preferably pyridyl, pyrimidyl, s-triazinyl or 1,2,4-triazinyl, in particular pyridyl or pyrimidyl.

Alkyl—as a group per se and as a structural element of other groups and compounds, such as of haloalkyl, alkoxy and alkylthio,—is, in each case taking into account the number of the carbon atoms included from case to case in the corresponding group or compound, either straight-chain, that is methyl, ethyl, propyl, butyl, pentyl, hexyl, heptyl or octyl, or branched, e.g. isopropyl, isobutyl, sec-butyl, tert-butyl, isopentyl, neopentyl or isohexyl.

Alkenyl—as a group per se and as a structural element of other groups and compounds—is, in each case taking into account the number of the carbon atoms and conjugated or isolated double bonds included from case to case in the corresponding group or compound, either straight-chain, e.g. allyl, 2-butenyl, 3-pentenyl, 1-hexenyl, 1-heptenyl, 1,3-hexadienyl or 1,3-octadienyl, or branched, e.g. isopropenyl, isobutenyl, isoprenyl, tert-pentenyl, isohexenyl, isoheptenyl or isooctenyl.

Alkynyl—as a group per se and as a structural element of other groups and compounds—is, in each case taking into account the number of the carbon atoms and conjugated or isolated double bonds included from case to case in the corresponding group or compound, either straight-chain, e.g. propargyl, 2-butynyl, 3-pentynyl, 1-hexynyl, 1-heptynyl, 3-hexen-1-ynyl, or 1,5-heptadien-3-ynyl, or branched, e.g. 3-methylbut-1-ynyl, 4-ethylpent-1-ynyl, 4-methylhex-2-ynyl or 2-methylhept-3-ynyl.

Cycloalkyl—as a group per se and as a structural element of other groups and compounds, such as of halocycloalkyl—is, in each case taking into account the number of the carbon atoms included from case to case in the corresponding group or compound, cyclopropyl, cyclobutyl, cyclopentyl, cyclohexyl, cycloheptyl or cyclooctyl.

Halogen—as a group per se and as a structural element of other groups and compounds—such as of haloalkyl, haloalkoxy and haloalkylthio—is fluorine, chlorine, bromine or iodine, in particular fluorine, chlorine or bromine, especially fluorine or chlorine.

Halogen-substituted carbon-containing groups and compounds, such as haloalkyl, halo-alkoxy or haloalkylthio, can be partially halogenated or perhalogenated, it being possible in the case of polysubstitution for the halogen substituents to be identical or different. Examples of haloalkyl—as a group per se and as a structural element of other groups and compounds—such as of haloalkoxy, or haloalkylthio, are methyl which is mono- to trisubstituted by fluorine, chlorine and/or bromine, such as CHF₂ or CF₃; ethyl which is mono- to pentasubstituted by fluorine, chlorine and/or bromine, such as CH₂CF₃, CF₂CF₃, CF₂CCl₃, CF₂CHCl₂, CF₂CHF₂, CF₂CFCl₂, CF₂CHBr₂, CF₂CHClF, CH₂CHBrF or CClFCHClF; propyl or isopropyl which is one mono- to heptasubstituted by fluorine, chlorine and/or bromine, such as CH₂CHBrCH₂Br, CF₂CHFCF₃, CH₂CF₂CF₃ or CH(CF₃)₂; butyl or one of its isomers which is mono- to nonasubstituted by fluorine, chlorine and/or bromine, such as CF(CF₃)CHFCF₃ or CH₂(CF₂)₂CF₃; pentyl or one of its isomers which is mono- to undecasubstituted by fluorine, chlorine and/or bromine, such as CF(CF₃)(CHF)₂CF₃ or CH₂(CF₂)CF₃; and hexyl or one of its isomers which is mono- to tridecasubstituted by fluorine, chlorine and/or bromine, such as (CH₂)₄CHBrCH₂Br, CF₂(CHF)₄CF₃, CH₂(CF₂)₄CF₃ or C(CF₃)₂(CHF)₂CF₃.

Alkoxy groups preferably have a chain length of 1 to 6 carbon atoms. Alkoxy is, for example, methoxy, ethoxy, propoxy, i-propoxy, n-butoxy, isobutoxy, sec-butoxy and tert-butoxy, and the isomers pentyloxy and hexyloxy; preferably methoxy and ethoxy. Haloalkoxy groups preferably have a chain length of 1 to 6 carbon atoms. Haloalkoxy is, for example, fluoromethoxy, difluoromethoxy, trifluoromethoxy, 2,2,2-trifluoroethoxy, 1,1,2,2-tetrafluoroethoxy, 2-fluoro-ethoxy, 2-chloroethoxy, 2,2-difluoroethoxy and 2,2,2-trichloroethoxy; preferably difluoromethoxy, 2-chloroethoxy and trifluoromethoxy.

Alkylthio groups preferably have a chain length of 1 to 6 carbon atoms. Alkylthio is, for example, methylthio, ethylthio, propylthio, isopropylthio, n-butylthio, isobutylthio, sec-butylthio or tert-butylthio, preferably methylthio and ethylthio.

Preferred embodiments within the context of the invention, taking into account the above-mentioned proviso, are:
(1) a compound of the formula (I), in which R₁ is aryl which is unsubstituted or mono- to pentasubstituted by R₇, where the substitutents in each case can be identical or different if their number is greater than 1; particularly aryl which is mono- to trisubstituted by R₇, where the substituents in each case can be identical or different if their number is greater than 1;
(2) a compound of the formula (I), in which R₂ is C₁-C₆alkyl, halo-C₁-C₆alkyl, aryl or heteroaryl which is in each case unsubstituted or mono- to polysubstituted by R₇, where the substituents can in each case be identical or different if their number is greater than 1; particularly C₁-C₆alkyl, halo-C₁-C₆alkyl or aryl which is unsubstituted or mono- to pentasubstituted by R₇, where the substituents can be identical or different if their number is greater than 1; very particularly aryl which is unsubstituted or mono- to trisubstituted by R₇, where the substituents can be identical or different if their number is greater than 1;
(3) a compound of the formula (I), in which R₃ is hydrogen or C₁-C₆alkyl; particularly hydrogen or C₁-C₄alkyl; very particularly hydrogen;
(4) a compound of the formula (I), in which R₄, R₅ and R₆ independently of one another are hydrogen, C₁-C₆alkyl, halo-C₁-C₆alkyl, C₁-C₆alkoxy, halo-C₁-C₆alkoxy, C₂-C₆alkenyl, C₂-C₆-alkynyl, C₃-C₆cycloalkyl; particularly hydrogen, C₁-C₄alkyl, halo-C₁-C₄alkyl or C₃-C₆cycloalkyl; very particularly hydrogen or C₁-C₂alkyl.
(5) a compound of the formula (I), in which R₇ is halogen, C₁-C₄alkyl, halo-C₁-C₄alkyl, C₁-C₄-alkoxy, halo-C₁-C₄alkoxy; aryl or phenylacetylenyl, in each case unsubstituted or mono- or polysubstituted, where the substituents are selected from the group consisting of halogen, C₁-C₆alkyl, halo-C₁-C₆alkyl, C₁-C₆alkoxy, halo-C₁-C₆alkoxy, and can in each case be identical or different if their number is greater than 1; particularly halogen, C₁-C₂alkyl, halo-C₁-C₂alkyl, C₁-C₂alkoxy, halo-C₁-C₂alkoxy; very particularly halogen or halo-C₁-C₂alkyl;
(6) a compound of the formula (I), in which R₈ is unsubstituted or mono- to trisubstituted aryl, where the substituents are selected from the group consisting of halogen, C₁-C₄alkyl, halo-C₁-C₄alkyl, C₁-C₄alkoxy and halo-C₁-C₄alkoxy, and can be identical or different if their number is greater than 1; particularly mono- to trisubstituted aryl, where the substituents are selected from the group consisting of halogen, C₁-C₂alkyl, halo-C₁-C₂alkyl, and halo-C₁-C₂alkoxy, and can be identical or different if their number is greater than 1; very particularly mono- or disubstituted aryl, where the substituents are selected from the group consisting of halogen and halo-C₁-C₂alkyl, and can be identical or different if their number is greater than 1;
(7) a compound of the formula (I), in which X is O or S; particularly 0;
(8) a compound of the formula (1), in which n is 1;
(9) a compound of the formula (1), in which R₁ is aryl which is unsubstituted or mono- or pentasubstituted by R₇, where the substituents can in each case be identical or different if their number is greater than 1; R₂ is C₁-C₆alkyl, halo-C₁-C₆alkyl, aryl or heteroaryl, in each case unsubstituted or mono- or polysubstituted by R₇, where the substituents can in each case be identical or different if their number is greater than 1; R₃ is hydrogen or C₁-C₆alkyl; R₄, R₅ and R₆ independently of one another are hydrogen, C₁-C₆alkyl, halo-C₁-C₆alkyl, C₁-C₆alkoxy, halo-C₁-C₆alkoxy, C₂-C₆alkenyl, C₂-C₆alkynyl, C₃-C₆cycloalkyl; R₇ is halogen, C₁-C₄alkyl, halo-C₁-C₄alkyl, C₁-C₄alkoxy, halo-C₁-C₄alkoxy, aryl or phenylacetylenyl, in each case unsubstituted or mono- or polysubstituted, where the substituents are selected from the group consisting of halogen, C₁-C₆alkyl, halo-C₁-C₆alkyl, C₁-C₆alkoxy, halo-C₁-C₆alkoxy, and in each case can be identical or different if their number is greater than 1; R₈ is unsubstituted or mono- to trisubstituted aryl, where the substituents are selected from the group consisting of halogen, C₁-C₄alkyl, halo-C₁-C₄alkyl, C₁-C₄alkoxy and halo-C₁-C₄alkoxy, and can be identical or different if their number is greater than 1; X is O or S; and n is 1;
(10) a compound of the formula (1), in which R₁ is aryl which is mono- or trisubstituted by R₇, where the substituents can in each case be identical or different if their number is greater than 1; R₂ is C₁-C₆alkyl, halo-C₁-C₆alkyl or aryl which is unsubstituted or mono- to pentasubstituted by R₇, where the substituents can be identical or different if their number is greater than 1; R₃ is hydrogen or C₁-C₄alkyl; R₄, R₅ and R₆ independently of one another are hydrogen, C₁-C₄alkyl, halo-C₁-C₄alkyl or C₃-C₆cycloalkyl; R₇ is halogen, C₁-C₂alkyl, halo-C₁-C₂alkyl, C₁-C₂alkoxy or halo-C₁-C₂alkoxy; R₈ is mono- to trisubstituted aryl, where the substituents are selected from the group consisting of halogen, C₁-C₂alkyl, halo-C₁-C₂alkyl, and halo-C₁-C₂alkoxy, and can be identical or different if their number is greater than 1; X is O; and n is 1;
(11) a compound of the formula (I), in which R₁ is aryl which is mono- to trisubstituted by R₇, where the substituents can in each case be identical or different if their number is greater than 1; R₂ is aryl which is unsubstituted or mono- to trisubstituted by R₇, where the substituents can in each case be identical or different if their number is greater than 1; R₃ is hydrogen; R₄, R₅ and R₆ independently of one another are hydrogen or C₁-C₂alkyl; R₇ is halogen or halo-C₁-C₂alkyl; R₈ is mono- or disubstituted aryl, where the substituents are selected from the group consisting of halogen and halo-C₁-C₂alkyl, and can be identical or different if their number is greater than 1; X is 0; and n is 1.

Particularly preferred compounds of the formula (I) for the purposes of the invention are those listed in tables 1 and 2 and very particularly preferably the compounds of the formula (I) mentioned in the synthesis examples.

A further subject of the invention is the process for the preparation of the compounds of the formula (I), in each case in free form or in salt form, characterized in that a compound of the formula 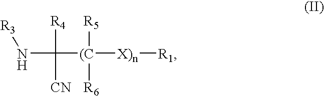
which is known or can be prepared in analogy to corresponding known compounds and in which R₁, R₃, R₄, R₅, R₆, X and n are as defined for the formula (I), is reacted with a compound of the formula 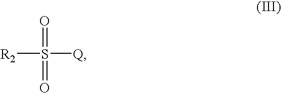
which is known or can be prepared in analogy to corresponding known compounds and in which R₂ is as defined for the formula (I) and Q is a leaving group, if appropriate in the presence of a basic catalyst, and in each case, if desired, a compound of the formula (I), in each case in free form or in salt form, obtainable according to the process or In another manner, is converted into another compound of the formula (I), a mixture of isomers obtainable according to the process is separated and the desired isomer is isolated and/or a free compound of the formula (I) obtainable according to the process is converted into a salt or a salt of a compound of the formula (I) obtainable according to the process is converted into the free compound of the formula (I) or into another salt.

For starting materials mentioned above and below, it applies with respect to their salts that what has been said above for salts of compounds of the formula (I) applies in an analogous manner.

The reactants can be reacted with one another as such, that is without addition of a solvent or diluent, e.g. In the melt. Usually, however, the addition of an inert solvent or diluent or of a mixture thereof is advantageous. Examples of such solvents or diluents which may be mentioned are: aromatic, aliphatic and alicyclic hydrocarbons and halogenated hydrocarbons, such as benzene, toluene, xylene, mesitylene, tetralin, chlorobenzene, dichlorobenzene, bromobenzene, petroleum ether, hexane, cyclohexane, dichloromethane, trichloromethane, tetrachloromethane, dichloroethane, trichloroethene or tetrachloroethene; ethers, such as diethyl ether, dipropyl ether, diisopropyl ether, dibutyl ether, tert-butyl methyl ether, ethylene glycol monomethyl ether, ethylene glycol monoethyl ether, ethylene glycol dimethyl ether, dimethoxydiethyl ether, tetrahydrofuran or dioxane; ketones, such as acetone, methyl ethyl ketone or methyl isobutyl ketone; amides, such as N,N-dimethylformamide, N,N-diethylformamide, N,N-dimethylacetamide, N-methylpyrrolidone or hexamethylphosphoramide; nitriles, such as acetonitrile or propionitrile; and sulphoxides, such as dimethyl sulphoxide.

Preferred leaving groups are halogens, in particular chlorine.

Suitable bases for facilitating the reaction are, for example, alkali metal or alkaline earth metal hydroxides, hydrides, amides, alkoxides, acetates, carbonates, dialkylamides or alkylsilylamides, alkylamines, alkylenediamines, free or N-alkylated, unsaturated or saturated, cycloalkylamines, basic heterocycles, ammonium hydroxides, and carbocyclic amines.

Examples which may be mentioned are sodium hydroxide, hydride, amide, methoxide, acetate, carbonate, potassium t-butoxide, hydroxide, carbonate, hydride, lithium diisopropylamide, potassium bis(trimethylsilyl)amide, calcium hydride, triethylamine, diisopropylamine, triethylenediamine, cyclohexylamine, N-cyclohexyl-N,N-dimethylamine, N,N-diethylaniline, pyridine, 4-(N,N-dlmethylamino)pyridine, quinuclidine, N-methylmorphollne, benzyltrimethylammonium hydroxide, and 1,5-diazabicyclo[5.4.0]undec-5-ene (DBU).

The reaction is advantageously carried out in a temperature range from approximately 0° C. to approximately +150° C., preferably from approximately 20° C. to approximately +100° C.

A further subject of the invention is the process for the preparation of the compounds of the formula (II), in each case in free form or in salt form, for example characterized in that a compound of the formula 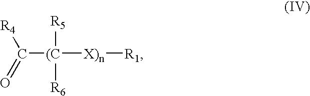
which is known or can be prepared in analogy to corresponding known compounds and in which R₁, R₄, R₅, R₆, X and n are as defined for the formula (I), is reacted with an inorganic or organic cyanide and a compound of the formula R₃—NH₂, which is known or can be prepared in analogy to corresponding known compounds and in which R₃ is as defined for the formula (I), and in each case, if desired, a compound of the formula (II), in each case in free form or in salt form, obtainable according to the invention or in another manner, is converted into another compound of the formula (II), a mixture of isomers obtainable according to the process is separated and the desired isomer is isolated and/or a free compound of the formula (II) obtainable according to the process is converted into a salt or a salt of a compound of the formula (II) obtainable according to the process is converted into the free compound of the formula (II) or into another salt.

Suitable cyanides are sodium cyanide, potassium cyanide, trimethylsilyl cyanide and acetone cyanohydrin.

Compounds of formula (I) obtainable in accordance with the process or by another method can be converted in a manner known per se into different compounds of formula (I) by replacing one or more substituents of the starting compound of formula (I) by (an)other substituent(s) according to the invention in customary manner.

Depending upon the reaction conditions and starting materials selected as suitable in each case, it is possible in a reaction step to replace only one substituent by another substituent according to the invention or it is possible in the same reaction step to replace a plurality of substituents by other substituents according to the invention.

Salts of compounds of formula (I) can be prepared in a manner known per se. For example, acid addition salts of compounds of formula (I) are obtained by treatment with a suitable acid or a suitable ion exchange reagent and salts with bases are obtained by treatment with a suitable base or a suitable ion exchange reagent.

Salts of compounds of formula (I) can be converted in customary manner into the free compounds of formula (I); acid addition salts can be converted, for example, by treatment with a suitable basic medium or a suitable ion exchange reagent and salts with bases, for example, by treatment with a suitable acid or a suitable ion exchange reagent.

Salts of compounds of formula (I) can be converted into different salts of compounds of formula (I) in a manner known per se; for example acid addition salts can be converted into different acid addition salts, for example by treatment of a salt of an inorganic acid, such as a hydrochloride, with a suitable metal salt, such as a sodium, barium or silver salt, of an acid, for example with silver acetate, in a suitable solvent in which an inorganic salt being formed, for example silver chloride, is insoluble and is therefore precipitated out from the reaction mixture.

Depending upon the procedure and/or the reaction conditions, the compounds of formula (I) having salt-forming properties can be obtained in free form or in the form of salts.

The compounds of formula (I) can also be obtained in the form of their hydrates and/or can include other solvents, for example any solvent that may have been used for the crystallisation of compounds in solid form.

The compounds of formulae (I) and (II) may be in the form of one of the possible isomers or in the form of a mixture thereof, for example depending upon the number of asymmetric carbon atoms and the absolute and relative configuration thereof, in the form of pure isomers, such as antipodes and/or diastereoisomers, or in the form of mixtures of isomers, such as mixtures of enantiomers, for example racemates, mixtures of diastereoisomers or mixtures of racemates; the invention relates both to the pure isomers and to all possible mixtures of isomers and this is to be understood accordingly hereinbefore and hereinafter, even when stereochemical details are not specifically mentioned in each case.

Mixtures of diastereoisomers and mixtures of racemates of compounds of formulae (I) and (II) obtainable in accordance with the process—depending upon the starting materials and procedures chosen—or by other means can be separated into the pure diastereoisomers or racemates in known manner on the basis of the physico-chemical differences between the constituents, for example by fractional crystallisation, distillation and/or chromatography.

Mixtures of enantiomers or racemates so obtainable can be separated into the optical antipodes by known methods, for example by recrystallisation from an optically active solvent, by chromatography on chiral adsorbents, for example high-pressure liquid chromatography (HPLC) on acetyl cellulose, with the aid of suitable microorganisms, by cleavage with specific immobilised enzymes, or via the formation of inclusion compounds, for example using chiral crown ethers, in which case only one enantiomer is complexed.

Pure diastereoisomers and enantiomers can be obtained not only by separation of corresponding mixtures of isomers but also, according to the invention, by generally known methods of diastereoselective or enantioselective synthesis, for example by carrying out the process according to the invention with starting materials that have appropriate stereochemistry.

It is advantageous to isolate or synthesise whichever isomer, for example enantiomer, or mixture of isomers, for example mixture of enantiomers, is biologically more active, insofar as the individual components have different biological activity.

In the process of the present invention there are preferably used those starting materials and intermediates which result in the compounds of formula (I) described at the beginning as being especially valuable.

The invention relates especially to the preparation process described in the Example.

The invention relates also to the novel starting materials and intermediates that are used according to the invention in the preparation of compounds of formula (I), to their use and to processes for the preparation thereof.

In the area of pest control, the compounds of formula (I) according to the invention are active ingredients exhibiting valuable preventive and/or curative activity with a very advantageous biocidal spectrum, even at low rates of concentration, while being well tolerated by warm-blooded organisms, fish and plants. The compounds are especially suitable for use in the area of controlling endo- and ecto-parasites of animals and plant-destructive insects and representatives of the order Acarina. The active ingredients according to the invention are effective against all or individual development stages of normally sensitive animal pests, but also of resistant animal pests, such as insects and representatives of the order Acarina. The pesticidal activity of the active ingredients according to the invention may manifest itself directly, i.e. In the mortality of the pests, which occurs immediately or only after some time, for example during moulting, or indirectly, for example in reduced oviposition and/or hatching rate, good activity corresponding to a mortality of at least 50 to 60%. The compounds of formula (I) are especially distinguished by an unusually long duration of action.

The said animal pests of plants include those mentioned in European Patent Application EP-A-736 252, page 5, line 55 to page 6, line 55. The pests mentioned therein are therefore included by reference in the subject matter of the present invention.

The compounds of formula (I) can also be used against pests affecting hygiene, especially of the order Diptera with the families Sarcophagidae, Anophilidae and Culicidae; and of the orders Orthoptera, Dictyoptera (e.g. the family Blattidae) and Hymenoptera (e.g. the family Formicidae).

The compounds of formula (I) also have long-lasting activity in the case of mites and insects that are parasites of plants. In the case of spider mites of the order Acarina, they are effective against eggs, nymphs and adults of Tetranychidae (Tetranychus spp. and Panonychus spp.).

They possess a high degree of activity in sucking insects of the order Homoptera, especially against pests of the families Aphididae, Delphacidae, Cicadellidae, Psyllidae, Loccidae, Diaspididae and Eriophydidae (e.g. rust mite on citrus fruit); of the orders Hemiptera, Heteroptera and Thysanoptera, and in phytophagous insects of the orders Lepidoptera, Coleoptera, Diptera and Orthoptera.

They are also suitable as a soil insecticide against pests in the soil.

The compounds according to the invention can be used to control, i.e. to inhibit or destroy, pests of the mentioned type occurring especially on plants, more especially on useful plants and ornamentals in agriculture, in horticulture and in forestry, or on parts of such plants, such as the fruits, blossoms, leaves, stems, tubers or roots, while in some cases parts of plants that grow later are still protected against those pests.

The compounds of formula (I) are therefore effective against all development stages of sucking and phytophagous insects on crops such as cereals, e.g. wheat, barley, rye, oats, rice, maize and sorghum; beet, such as sugar beet and fodder beet; fruit, e.g. pomes, stone fruit and soft fruit, such as apples, pears, plums, peaches, almonds, cherries and berries, e.g. strawberries, raspberries and blackberries; leguminous plants, such as beans, lentils, peas and soybeans; oil plants, such as rape, mustard, poppy, olives, sunflowers, coconut, castor oil, cocoa and groundnuts; cucurbitaceae, such as marrows, cucumbers and melons; fibre plants, such as cotton, flax, hemp and jute; citrus fruits, such as oranges, lemons, grapefruit and mandarins; vegetables, such as spinach, lettuce, asparagus, cabbages, carrots, onions, tomatoes, potatoes and paprika; lauraceae, such as avocado, cinnamon and camphor; and tobacco, nuts, coffee, aubergines, sugar cane, tea, pepper, vines, hops, bananas, natural rubber plants and ornamentals.

The compounds of formula (I) are also effective against plant-nematodes of the species Meloidogyne, Heterodera, Pratylenchus, Ditylenchus, Radopholus, Rizoglyphus and others.

In the context of the present invention, ectoparasites occurring as parasites on warm-blooded organisms are understood to mean especially insects, mites and ticks. Included are insects of the orders: Lepidoptera, Coleoptera, Homoptera, Heteroptera, Diptera, Thysanoptera, Orthoptera, Anoplura, Siphonaptera, Mallophaga, Thysanura, Isoptera, Psocoptera and Hymenoptera. Special mention may be made, however, of ectoparasites that trouble humans and animals and transmit pathogens, for example flies, such as Musca domestica, Musca vetustissima, Musca autumnalis, Fannia canicularis, Sarcophaga camaria, Lucilia cuprina, Hypoderma bovis, Hypoderma lineatum, Chrysomyia chloropyga, Dermatobia hominis, Cochliomyla hominivorax, Gasterophilus intestinalis, Oestrus ovis, Stomoxys calcitrans, Haematobia irritans and midges (Nematocera), such as Culicidae, Simuliidae, Psychodidae, and also bloodsucking parasites, for example fleas, such as Ctenocephalides felis and Ctenocephalides canis (cat and dog fleas), Xenopsylla cheopis, Pulex irritans, Dermatophilus penetrans, lice, such as Damalina ovis, Pediculus humanis, stable flies and horse flies (Tabanidae), Haematopota spp., such as Haematopota pluvialis, Tabanidea spp., such as Tabanus nigrovittatus, Chrysopsinae spp., such as Chrysops caecutiens, tsetse flies, such as Glossinia species, biting insects, especially cockroaches, such as Blatella germanica, Blatta orientalis, Periplaneta americana, mites, such as Dermanyssus gallnae, Sarcoptes scabiei, Psoroptes ovis and Psorergates spp. and, not least, ticks. The latter belong to the order Acarina. Known examples of ticks are, for example, Boophilus, Amblyomma, Anocentor, Dermacentor, Haemaphysalis, Hyalomma, Ixodes, Rhipicentor, Margaropus, Rhipicephalus, Argas, Otobius and Ornithodoros and the like, which preferentially infest warm-blooded animals, including farm animals, such as cows, pigs, sheep and goats, poultry, such as hens, turkeys and geese, animals bred for their fur, such as mink, fox, chinchillas, rabbits and the like, and domestic animals, such as cats and dogs, but also humans.

In addition, the compounds of formula (I) are especially effective against helminths, among which the endoparasitic nematodes and trematodes may be the cause of serious diseases of mammals and poultry, for example of sheep, pigs, goats, cattle, horses, donkeys, dogs, cats, guinea-pigs and ornamental birds. Typical nematodes in that indication are: Haemonchus, Trichostrongylus, Ostertagia, Nematodirus, Cooperia, Ascaris, Bunostonum, Oesophagostonum, Chabertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. Among the trematodes special mention should be made of the family of the Fasciolideae, especially Fasciola hepatica. The special advantage of the compounds of formula (I) is their efficacy against such parasites that are resistant to benzimidazole-based active ingredients.

Certain Nematodirus, Cooperia and Oesophagostonum species attack the intestinal tract of the host animal whereas others of the species Haemonchus and Ostertagia parasitise in the stomach and those of the species Dictyocaulus parasitise in pulmonary tissue. Parasites of the families Filariidae and Setariidae are found in the internal cell tissue and the organs, for example the heart, the blood vessels, the lymph vessels and the subcutaneous tissue. In this context special mention should be made of dog heartworm, Dirofilaria immitis. The compounds of formula (I) are highly effective against those parasites.

Furthermore, the compounds of formula (I) are suitable for controlling parasites that are pathogens of humans, among which, as typical representatives occurring in the digestive tract, mention should be made of those of the species Ancylostoma, Necator, Ascaris, Strongyloldes, Trichinella, Capillaria, Trichuris and Enterobius. The compounds of the present invention are also effective against parasites of the species Wuchereria, Brugia, Onchocerca and Loa from the family of the Filariidae, which occur in the blood, in tissue and various organs, and also against Dracunculus and parasites of the species Strongyloides and Trichinella, which infect especially the gastrointestinal tract.

In the area of crop protection, the compounds of formula (I) are used in unmodified form or, preferably, together with the adjuvants conventionally employed in formulation technology and can therefore be formulated in known manner e.g. into emulsifiable concentrates, directly dilutable solutions, dilute emulsions, soluble powders, granules, and also encapsulations in polymer substances. As with the nature of the compositions, the methods of application are selected in accordance with the intended objectives and the prevailing circumstances.

The invention relates also to pesticides, such as emulsifiable concentrates, suspension concentrates, directly sprayable or dilutable solutions, coatable pastes, dilute emulsions, wettable powders, soluble powders, dispersible powders, wettable powders, dusts, granules or encapsulations in polymer substances, comprising at least one of the active ingredients of the invention, the type of formulation being chosen in accordance with the intended objectives and prevailing circumstances. They are prepared in known manner, e.g. by homogeneously mixing and/or grinding the active ingredients with extenders, for example with solvents, solid carriers, and optionally surface-active compounds (surfactants).

The active ingredient is used in those compositions in pure form: a solid active ingredient, for example, in a specific particle size, or preferably together with at least one of the adjuvants customary in formulation technology, such as extenders, for example solvents or solid carriers, or surface-active compounds (surfactants).

As formulation adjuvants there are used, for example, solid carriers, solvents, stabilisers, “slow release” adjuvants, dyes and optionally surface-active substances (surfactants). Suitable carriers and adjuvants include all those substances customarily used in crop protection products, especially in snail and slug control products. Suitable adjuvants, such as solvents, solid carriers, surface-active compounds, non-ionic surfactants, cationic surfactants, anionic surfactants and other adjuvants in the compositions used according to the invention, include e.g. those described in EP-A-736 252, page 7, line 51, to page 8, line 39. They are included by reference in the subject matter of the present invention.

The compositions for use in crop protection generally comprise from 0.1 to 99% by weight, especially from 0.1 to 95% by weight, of active ingredient and from 1 to 99.9% by weight, especially from 5 to 99.9% by weight, of at least one solid or liquid adjuvant, it generally being possible for from 0 to 25% by weight, especially from 0.1 to 20%, of the compositions to consist of surfactants. Whereas commercial products will preferably be formulated as concentrates, the end user will normally employ dilute formulations, which have much lower active ingredient concentrations. Preferred compositions used in crop protection have especially the following compositions (%=% by weight):

| | | |
|---|---|---|
| Emulsifiable concentrates: | | |
| active ingredient: | 1 to 95%, | preferably 5 to 20% |
| surfactant: | 1 to 30%, | preferably 10 to 20% |
| solvent: | 5 to 98%, | preferably 70 to 85% |
| Dusts: | | |
| active ingredient: | 0.1 to 10%, | preferably 0.1 to 1% |
| solid carrier: | 99.9 to 90%, | preferably 99.9 to 99% |
| Suspension concentrates: | | |
| active ingredient: | 5 to 75%, | preferably 10 to 50% |
| water: | 94 to 24%, | preferably 88 to 30% |
| surfactant: | 1 to 40%, | preferably 2 to 30% |
| Wettable powders: | | |
| active ingredient: | 0.5 to 90%, | preferably 1 to 80% |
| surfactant: | 0.5 to 20%, | preferably 1 to 15% |
| solid carrier: | 5 to 99%, | preferably 15 to 98% |
| Granules: | | |
| active ingredient: | 0.5 to 30%, | preferably 3 to 15% |
| solid carrier: | 99.5 to 70%, | preferably 97 to 85% |

The anthelmintic compositions according to the invention for the control of animal parasites in and on warm-blooded organisms generally comprise from 0.1 to 99% by weight, especially from 0.1 to 95% by weight, of compound of formula (I), from 99.9 to 1% by weight, especially from 99.8 to 5% by weight, of a solid or liquid auxiliary, which includes from 0 to 25% by weight, especially from 0.1 to 25% by weight, of a surfactant.

Preferred forms of administration for use in warm-blooded organisms for controlling helminths Include solutions, emulsions, suspensions (drenches), feed additives, powders, tablets including effervescent tablets, boli, capsules, micro-encapsulations and pour-on formulations, it being necessary to ensure that the formulation adjuvants are physiologically tolerable.

Suitable solvents in the use of formulations for controlling animal parasites are, for example: alcohols, e.g. ethanol, propanol or butanol, and glycols and ethers and esters thereof, e.g. propylene glycol, dipropylene glycol ether, ethylene glycol, ethylene glycol monomethyl or monoethyl ether, ketones, e.g. cyclohexanone, isophorone or diacetone alcohol, strong polar solvents, e.g. N-methyl-2-pyrrolidone, dimethyl sulfoxide or dimethylformamide or water, vegetable oils, e.g. rapeseed oil, castor oil, coconut oil or soybean oil, and also, where appropriate, silicone oils.

Suitable binders for tablets and boli are chemically modified natural polymeric substances soluble in water or in alcohol, for example starch, cellulose or protein derivatives (e.g. methylcellulose, carboxymethylcellulose, ethyl hydroxyethylcellulose, proteins such as zein, gelatin and the like) and synthetic polymers, e.g. polyvinyl alcohol, polyvinylpyrrolidone etc. Tablets also comprise fillers (e.g. starch, microcrystalline cellulose, sugar, lactose etc.), lubricants and disintegrants.

When the anthelmintic compositions are in the form of feed concentrates, there are used as carriers, for example, performance feeds, feed grains or protein concentrates. Such feed concentrates or compositions can also comprise, besides the active ingredients, auxiliaries, vitamins, antibiotics, chemotherapeutic agents or other pesticides, especially bacteriostatics, fungistatics, coccidiostatics and also hormone preparations, substances having an anabolic action or substances that promote growth, that influence the quality of meat of slaughtered animals or that are useful in some other way for the organism. When the compositions or the compounds of formula (I) contained therein are added directly to the feed or drinking troughs, the final feed or drinking troughs contain the active ingredients in a concentration of preferably from 0.0005 to 0.02% by weight (5-200 ppm).

The compounds of formula (I) according to the invention can be used alone or in combination with other biocides. For example, in order to enhance the effect they can be combined with pesticides having the same direction of action or, in order to broaden the spectrum of activity, they can be combined with substances having a different direction of action. It can also be of advantage to add so-called ‘repellents’. Where it is desired to extend the spectrum of activity to endoparasites, e.g. worms, the compounds of formula (I) are advantageously combined with substances having endoparasiticidal properties. They can, of course, also be used in combination with anti-bacterial agents. Since the compounds of formula (I) are “adulticides”, i.e. since they are effective especially against the mature stages of target parasites, the addition of pesticides which are more effective against the juvenile stages of the parasites may be very advantageous, since in this way the major portion of those parasites that cause large-scale economic damage will be reached, significantly contributing, moreover, to the avoidance of the formation of resistance. Some combinations may also result in synergistic effects, that is to say the overall amount of active substance used can be reduced, which is desirable from an ecological standpoint. Preferred groups of combination partners and especially preferred combination partners are mentioned hereinbelow; the combinations may comprise, in addition to a compound of formula (I), one or more of such partners.

Suitable mixing partners for use in crop protection and for use in controlling endo- and ecto-parasites on warm-blooded organisms are biocides, for example the insecticides and acaricides mentioned hereinbelow and sufficiently known to the person skilled in the art which have a different mechanism of action, for example chitin synthesis inhibitors, growth regulators; active ingredients that act in the same manner as juvenile hormones; active ingredients that act as adulticides; broad-spectrum insecticides, broad-spectrum acaricides, and nematicides; and also the sufficiently known anthelmintics, and substances repelling insects and/or Acarina, the said repellents and detachers.

Examples of suitable insecticides and acaricides are azamethiphos; chlorfenvinphos; cypermethrin, cypermethrin high-cis; cyromazine; diafenthiuron; diazinon; dichlorvos; dicrotophos; dicyclanil; fenoxycarb; fluazuron; furathiocarb; isazofos; jodfenphos; kinoprene; lufenuron; methacriphos; methidathion; monocrotophos; phosphamidon; profenofos; diofenolan; a substance obtainable from the Bacillus thuringiensis strain GC91 or from the strain NCTC11821; pymetrozine; bromopropylate; methoprene; disulfuton; quinalphos; tau-fluvalinate; thiocyclam; thiometon; aldicarb; azinphos-methyl; benfuracarb; bifenthrin; buprofezin; carbofuran; dibutylaminothio; cartap; chlorfluazuron; chlorpyrifos; cyfluthrin; lambda-cyhalothrin; alpha-cypermethrin; zeta-cypermethrin; deltamethrin; diflubenzuron; endosulfan; ethiofencarb; fenitrothion; fenobucarb; fenvalerate; formothion; methiocarb; heptenophos; imidacloprid; isoprocarb; methamidophos; methomyl; mevinphos; parathion; parathion-methyl; phosalone; pirimicarb; propoxur; teflubenzuron; terbufos; triazamate; fenobucarb; tebufenozide; fipronil; beta-cyfluthrin; silafluofen; fenpyroximate; pyridaben; fenazaquin; pyriproxyfen; pyrimidifen; nitenpyram; acetamiprid; avermectin B₁ (abamectin); emamectin; emamectin benzoate; spinosad; a plant extract that is active against insects; a preparation comprising nematodes which is active against insects; a preparation obtainable from Bacillus subtilis; a preparation comprising fungi which is active against insects; a preparation comprising viruses which is active against insects; chlorfenapyr; acephate; acrinathrin; alanycarb; alphamethrin; amitraz; Az 60541; azinphos A; azinphos M; azocyclotin; bendiocarb; bensultap; beta-cyfluthrin; BPMC; brofenprox; bromophos A; bufencarb; butocarboxin; butylpyridaben; cadusafos; carbaryl; carbophenothion; chloethocarb; chlorethoxyfos; chlormephos; cis-resmethrin; clocythrin; clofentezine; cyanophos; cycloprothrin; cyhexatin; demeton M; demeton S; demeton-5-methyl; dichlofenthion; dicliphos; diethion; dimethoate; dimethylvinphos; dioxathion; edifenphos; esfenvalerate; ethion; ethofenprox; ethoprophos; etrimphos; fenamiphos; fenbutatin oxide; fenbthiocarb; fenpropathrin; fenpyrad; fenthion; fluazinam; flucycloxuron; flucythrinate; flufenoxuron; flufenprox; fonophos; fosthiazate; fubfenprox; HCH; hexaflumuron; hexythiazox; IKI-220; iprobenfos; isofenphos; isoxathion; ivermectin; malathion; mecarbam; mesulfenphos; metaldehyde; metolcarb; milbemectin; moxidectin; naled; NC 184; omethoate; oxamyl; oxydemeton M; oxydeprofos; permethrin; phenthoate; phorate; phosmet; phoxim; pirimiphos M; pirimiphos E; promecarb; propaphos; prothiofos; prothoate; pyrachlophos; pyrada-phenthion; pyresmethrin; pyrethrum; tebufenozide; salithion; sebufos; sulfotep; sulprofos; tebufenpyrad; tebupirimphos; tefluthrin; temephos; terbam; tetrachlorvinphos; thiacloprid; thiafenox; thiamethoxam; thiodicarb; thiofanox; thionazin; thuringiensin; tralomethrin; triarthene; triazophos; triazuron; trichlorfon; triflumuron; trimethacarb; vamidothion; xylylcarb; YI 5301/5302; zetamethrin; DPX-MP062-indoxacarb; methoxyfenozide; bifenazate; XMC (3,5-xylyl methylcarbamate); or the fungus pathogen Metarhizium Anisopliae. The mentioned mixing partners are very well known to persons skilled In the art. Most are described in various editions of The Pesticide Manual, The British Crop Protection Council, London, while others are described in various editions of The Merck Index, Merck & Co., Inc., Rahway, N.J., USA, or in the patent literature.

Examples of suitable anthelmintics that can be added to the compositions are mentioned hereinbelow, a number of the examples thereof having, in addition to anthelmintic activity, also an insecticidal and acaricidal activity, some of them already being mentioned in the list above:
(A1) praziguantel=2-cyclohexylcarbonyl-4-oxo-1,2,3,6,7,11 b-hexahydro-4H-pyrazino[2,1-α]isoquinoline
(A2) closantel=3,5-diiodo-N-[5-chloro-2-methyl-4-(a-cyano-4-chlorobenzyl)phenyl]salicylamide
(A3) triclabendazole=5-chloro-6-(2,3-dichlorophenoxy)-2-methylthio-1H-benzimidazole
(A4) levamisol=L-(−)-2,3,5,6-tetrahydro-6-phenylimidazo[2,1b]thiazole
(A5) mebendazole=(5-benzoyl-1H-benzimidazol-2-yl)carbamic acid methyl ester
(A6) omphalotin=a macrocyclic fermentation product of the fungus Omphalotus olearius described in WO 97/20857
(A7) abamectin=avermectin B1
(A8) ivermectin=22,23-dihydroavermectin B1
(A9) moxidectin=5-O-demethyl-28-deoxy-25-(1,3-dimethyl-1-butenyl)-6,28-epoxy-23-(methoxyimino)-milbemycin B
(A10) doramectin=25-cyclohexyl-5-O-demethyl-25-de(1-methylpropyl)-avermectin Ala
(A11) milbemectin=mixture of milbemycin A3 and milbemycin A4
(A12) milbemycinoxim=5-oxime of milbemectin

Examples of suitable repelling substances (repellents and detachers) are for instance:
(R1) DEET (N,N-diethyl-m-toluamide)
(R2) KBR 3023 N-butyl-2-oxycarbonyl-(2-hydroxy)-piperidine
(R3) cymiazol=N-2,3-dihydro-3-methyl-1,3-thiazol-2-ylidene-2,4-xylidine

Given what has been said above, a further substantial aspect of the present invention relates to combination preparations for the control of parasites on warm-blooded organisms, which combination preparations comprise, in addition to a compound of formula (1), at least one further active ingredient having the same direction of action or a different direction of action and at least one physiologically tolerable carrier. The present invention is not limited to two-component combinations.

The compositions according to the invention can be administered to the animals being treated by topical, peroral, parenteral or subcutaneous means, the compositions being in the form of solutions, emulsions, suspensions (drenches), powders, tablets, boli, capsules and pour-on formulations.

The pour-on or spot-on method comprises applying the compound of formula (I) to a locally defined area of the skin or coat, advantageously on the back of the neck or the backbone of the animal. This is carried out, for example, by applying a swab or spray of the pour-on or spot-on formulation to a relatively small area of the coat from where the active ingredient becomes distributed over a wide area of the coat almost automatically as a result of the spreading constituents of the formulation assisted by the movements of the animal.

Pour-on and spot-on formulations advantageously comprise carriers that promote rapid distribution over the surface of the skin or in the coat of the host animal and are generally termed spreading oils. There are suitable, for example, oily solutions; alcoholic and isopropanolic solutions, e.g. solutions of 2-octyl-dodecanol or oleyl alcohol; solutions in esters of monocarboxylic acids, such as isopropyl myristate, isopropyl palmitate, lauric acid oxalic ester, oleic acid oleyl ester, oleic acid decyl ester, hexyl laurate, oleyl oleate, decyl oleate, capric acid esters of saturated fatty alcohols of chain length C₁₂-C₁₈; solutions of esters of dicarboxylic acids, such as dibutyl phthalate, diisopropyl isophthalate, adipic acid diisopropyl ester, di-n-butyl adipate or solutions of esters of aliphatic acids, e.g. glycols. It may be advantageous for a dispersant known from the pharmaceutical or cosmetic industry also to be present. Examples are 2-pyrrolidone, 2-(N-alkyl)pyrrolidone, acetone, polyethylene glycol and its ethers and esters, propylene glycol or synthetic triglycerides.

The oily solutions include e.g. vegetable oils, such as olive oil, groundnut oil, sesame oil, pine oil, linseed oil and castor oil. The vegetable oils may also be in epoxidised form. It is also possible to use paraffins and silicone oils.

Generally a pour-on or spot-on formulation will contain from 1 to 20% by weight of a compound of formula (I), from 0.1 to 50% by weight dispersant and from 45 to 98.9% by weight solvent.

The pour-on or spot-on method can be used especially advantageously for herd animals, such as cattle, horses, sheep and pigs, where it is difficult or time-consuming to treat all the animals orally or via injection. By virtue of its simplicity, this method can of course also be used for all other animals, including individual domestic animals and pets, and is very popular with the keepers of the animals because it can often be carried out without the expert assistance of a veterinary surgeon.

Whereas commercial products will preferably be formulated as concentrates, the end user will normally employ dilute formulations.

Such formulations may also comprise further ingredients, such as stabilisers, antifoams, viscosity regulators, binders and tackifiers as well as other active ingredients for obtaining special effects.

The present invention relates also to such anthelmintic compositions employed by the end user.

In each of the methods according to the invention for controlling pests and in each of the pesticidal compositions according to the invention, the active ingredients of formula (I) can be used in any of their spatial configurations or mixtures thereof.

The invention also encompasses a method for the prophylactic protection of warm-blooded organisms, especially of productive livestock, domestic animals and pets, against parasitic helminths, which method comprises administering to the animals the active ingredient of formula (I) or active ingredient formulations prepared therefrom as an additive to the feed or to the drinking troughs or in solid or liquid form orally, by injection or parenterally. The invention also encompasses the compounds of formula (I) according to the invention for use in one of the mentioned methods.

The Examples that follow serve merely to illustrate the invention without limiting the invention, the expression active ingredient denoting one of the substances listed in the Tables.

Preferred formulations for use in the control of parasites on warm-blooded organisms have the following compositions: (%=% by weight)

1. Granules

| | a) | b) |
|---|---|---|
| active ingredient from Tables 1 or 2 | 5% | 10% |
| kaolin | 94% | — |
| highly disperse silicic acid | 1% | — |
| attapulgite | — | 90% |

The active ingredient is dissolved in methylene chloride and sprayed onto the carrier, and the solvent is then evaporated off in vacuo. Such granules can be mixed into the animal feed.

2. Granules

| | |
|---|---|
| active ingredient from Tables 1 or 2 | 3% |
| polyethylene glycol (MW 200) | 3% |
| kaolin | 94% |

(MW = molecular weight)

The finely ground active ingredient is uniformly applied, in a mixer, to the kaolin moistened with polyethylene glycol. Non-dusty coated granules are obtained in this manner.

3. Tablets and boli

| | | |
|---|---|---|
| I | active ingredient from Tables 1 or 2 | 33.00% |
| | methylcellulose | 0.80% |
| | highly disperse silicic acid | 0.80% |
| | maize starch | 8.40% |
| II | crystalline lactose | 22.50% |
| | maize starch | 17.00% |
| | microcrystalline cellulose | 16.50% |
| | magnesium stearate | 1.00% |

I Methylcellulose is stirred into water. After the material has swelled, silicic acid is stirred in and the mixture is homogeneously suspended. The active ingredient and maize starch are mixed. The aqueous suspension is incorporated into the resulting mixture and kneaded to a dough. The mass thereby obtained is granulated through a 12 M sieve and dried.
II All 4 excipients are mixed well.
III The premixes obtained according to 1 and 11 are mixed and compressed into tablets or boli.

4. Injectable formulations

A. Oily vehicle (slow release)

| | | | |
|---|---|---|---|
| 1. | active ingredient from Tables 1 or 2 | 0.1-1.0 | g |
| | groundnut oil | ad 100 | ml |
| 2. | active ingredient from Tables 1 or 2 | 0.1-1.0 | g |
| | sesame oil | ad 100 | ml |

Preparation: The active ingredient is dissolved in a portion of the oil with stirring and optionally with gentle heating, and after cooling the solution is made up to the desired volume and sterile-filtered through a suitable 0.22 micron membrane filter.

B. Water-miscible solvent (medium rate of release)

| | | |
|---|---|---|
| active ingredient from Tables 1 or 2 | 0.1-1.0 | g |
| 4-hydroxymethyl-1,3-dioxolane (glycerol formal) | 40 | g |
| 1,2-propanediol | ad 100 | ml |
| an active ingredient from Table 1 | 0.1-1.0 | g |
| glycerol dimethyl ketal | 40 | g |
| 1,2-propanediol | ad 100 | ml |

Preparation: The active ingredient is dissolved in a portion of the solvent with stirring, and the solution is made up to the desired volume and sterile-filtered through a suitable 0.22 micron membrane filter.

C. Aqueous solubilisate (rapid release)

| | | | |
|---|---|---|---|
| 1. | active ingredient from Tables 1 or 2 | 0.1-1.0 | g |
| | polyethoxylated castor oil (40 ethylene oxide | 10 | g |
| | units) | | |
| | 1,2-propanediol | 20 | g |
| | benzyl alcohol | 1 | g |
| | aqua ad inject. | ad 100 | ml |
| 2. | active ingredient from Tables 1 or 2 | 0.1-1.0 | g |
| | polyethoxylated sorbitan monooleate (20 ethylene | 8 | g |
| | oxide units) | | |
| | 4-hydroxymethyl-1,3-dioxolane (glycerol formal) | 20 | g |
| | benzyl alcohol | 1 | g |
| | aqua ad inject. | ad 100 | ml |

Preparation: The active ingredient is dissolved in the solvents and the surfactant, and the solution is made up to the desired volume with water. Sterile-filtration is then carried out through a suitable membrane filter of 0.22 micron pore diameter.

5. Pour-on

| | | |
|---|---|---|
| A. | | |
| active ingredient from Tables 1 or 2 | 5 | g |
| isopropyl myristate | 10 | g |
| isopropanol | ad 100 | ml |
| B. | | |
| active ingredient from Tables 1 or 2 | 2 | g |
| hexyl laurate | 5 | g |
| medium-chain triglycerides | 15 | g |
| ethanol | ad 100 | ml |
| C. | | |
| active ingredient from Tables 1 or 2 | 2 | g |
| oleyl oleate | 5 | g |
| N-methyl-pyrrolidone | 40 | g |
| isopropanol | ad 100 | ml |

The aqueous systems may preferably be used also for oral and/or intraruminal administration.

The compositions may also comprise further ingredients such as stabilisers, e.g. vegetable oils and epoxidised vegetable oils (epoxidised coconut oil, rapeseed oil or soybean oil), antifoams, e.g. silicone oil, preservatives, viscosity regulators, binders, tackifiers and fertilisers as well as other active ingredients for obtaining special effects.

It is also possible to add to the described compositions further biologically active substances or additives that have neutral behaviour towards the compounds of formula (I) and have no adverse effect on the host animal to be treated, and also mineral salts or vitamins.

The compositions according to the invention are prepared in known manner, in the absence of adjuvants, for example by grinding, sieving and/or compressing a solid active ingredient or mixture of active ingredients, for example to a specific particle size, or in the presence of at least one adjuvant, for example by intimately mixing and/or grinding the active ingredient or mixture of active ingredients with the adjuvant(s). The invention relates also to those processes for the preparation of the compositions according to the invention and to the use of the compounds of the formula (I) in the preparation of those compositions.

The invention relates also to the methods of application of the compositions, i.e. the methods of controlling pests of the mentioned type, such as spraying, atomising, dusting, coating, dressing, scattering or pouring, which are selected in accordance with the intended objectives and prevailing circumstances, and to the use of the compositions for controlling pests of the mentioned type. Typical rates of concentration are from 0.1 to 1000 ppm, preferably from 0.1 to 500 ppm, of active ingredient. The rates of application per hectare are generally from 1 to 2000 g of active ingredient per hectare, especially from 10 to 1000 g/ha, preferably from 20 to 600 g/ha.

A preferred method of application in the area of crop protection is application to the foliage of the plants (foliar application), the number of applications and the rate of application depending on the risk of infestation by the pest in question. However, the active ingredient can also penetrate the plants through the roots (systemic action) if the locus of the plants is impregnated with a liquid formulation or if the active ingredient is incorporated in solid form into the locus of the plants, for example into the soil, e.g. in granular form (soil application). In paddy rice crops, such granules may be applied in metered amounts to the flooded rice field.

The compositions according to the invention are also suitable for protecting plant propagation material, including genetically modified propagation material, e.g. seed material, such as fruit, tubers or grains, or plant cuttings, from animal pests. The propagation material can be treated with the formulation before planting: seed, for example, can be dressed before being sown. The compounds according to the invention can also be applied to grains (coating), either by impregnating the grains with a liquid formulation or by coating them with a solid formulation. The formulation can also be applied to the planting site when the propagation material is being planted, for example to the seed furrow during sowing. The invention relates also to those methods of treating plant propagation material and to the plant propagation material thus treated.

The following Examples serve to illustrate the invention. They do not limit the invention. The symbol ‘h’ stands for ‘hour’.

### PREPARATION EXAMPLES

### Preparation of N-(1-cyano-1-[2,3-dichlorophenoxymethyl]ethyl)-C-phenylmethanesulphonamide

a) A mixture of 5 g of 2,3-dichlorophenol, 4 g of 2-chloroacetone, 4.7 g of anhydrous potassium carbonate and 450 mg of potassium iodide is refluxed in 50 ml of acetone for 6 h, then cooled to room temperature and filtered. The filtrate is evaporated. 1-(2,3-Dichloro-phenoxy)acetone is obtained in this way.

b) 6.5 g of 1-(2,3-dichlorophenoxy)acetone, 1.76 g of sodium cyanide and 2.4 g of ammonium chloride are added to a solution of 30 ml of 25% ammonia and the mixture is stirred at room temperature for 24 h. The crude product is then extracted from the mixture using ethyl acetate, and the organic phase is separated off, washed with water and saturated sodium chloride solution, dried using magnesium sulphate and evaporated. 2-Amino-3-(2,3-dichlorophenoxy)-2-methylpropionitrile is thus obtained.

c) 5.2 g of α-toluenesulphonyl chloride are added to a mixture of 6.6 g of 2-amino-3-(2,3-dichlorophenoxy)-2-methylpropionitrile and 4.86 g of ethyldiisopropylamines in 50 ml of methylene chloride and the mixture is stirred at room temperature for 24 h. The crude product is then extracted from the mixture with ethyl acetate, and the organic phase is separated off, washed with aqueous sodium bicarbonate and saturated sodium chloride solution, dried using magnesium sulphate and evaporated. The residue is purified by high-pressure column chromatography on silica gel using hexane/ethyl acetate (2:1). The title compound is thus obtained.

The substances mentioned in the table below can also be prepared analogously to the procedures described above. The values of the melting points are indicated in ° C.

TABLE 1

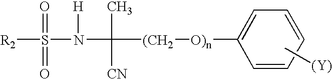

| No. | R₂ | n | (Y) | phys. Data |
|---|---|---|---|---|
| 1.1 | CH₃ | 0 | H | |
| 1.2 | CH₃ | 0 | 2-F | |
| 1.3 | CH₃ | 0 | 3-F | |
| 1.4 | CH₃ | 0 | 4-F | |
| 1.5 | CH₃ | 0 | 2-Cl | |
| 1.6 | CH₃ | 0 | 3-Cl | |
| 1.7 | CH₃ | 0 | 4-Cl | |
| 1.8 | CH₃ | 0 | 2,3-Cl₂ | |
| 1.9 | CH₃ | 0 | 2,4-Cl₂ | |
| 1.10 | CH₃ | 0 | 2,5-Cl₂ | |
| 1.11 | CH₃ | 0 | 2,6-Cl₂ | |
| 1.12 | CH₃ | 0 | 3,4-Cl₂ | |
| 1.13 | CH₃ | 0 | 3,5-Cl₂ | |
| 1.14 | CH₃ | 0 | 3-CF₃ | |
| 1.15 | CH₃ | 0 | 4-CF₃ | |
| 1.16 | CH₃ | 1 | H | |
| 1.17 | CH₃ | 1 | 2-F | |
| 1.18 | CH₃ | 1 | 3-F | |
| 1.19 | CH₃ | 1 | 4-F | |
| 1.20 | CH₃ | 1 | 2-Cl | |
| 1.21 | CH₃ | 1 | 3-Cl | |
| 1.22 | CH₃ | 1 | 4-Cl | |
| 1.23 | CH₃ | 1 | 2,3-Cl₂ | |
| 1.24 | CH₃ | 1 | 2,4-Cl₂ | |
| 1.25 | CH₃ | 1 | 2,5-Cl₂ | |
| 1.26 | CH₃ | 1 | 2,6-Cl₂ | |
| 1.27 | CH₃ | 1 | 3,4-Cl₂ | |
| 1.28 | CH₃ | 1 | 3,5-Cl₂ | |
| 1.29 | CH₃ | 1 | 3-CF₃ | |
| 1.30 | CH₃ | 1 | 4-CF₃ | |
| 1.31 | C₂H₅ | 0 | H | |
| 1.32 | C₂H₅ | 0 | 2-F | |
| 1.33 | C₂H₅ | 0 | 3-F | |
| 1.34 | C₂H₅ | 0 | 4-F | |
| 1.35 | C₂H₅ | 0 | 2-Cl | |
| 1.36 | C₂H₅ | 0 | 3-Cl | |
| 1.37 | C₂H₅ | 0 | 4-Cl | |
| 1.38 | C₂H₅ | 0 | 2,3-Cl₂ | |
| 1.39 | C₂H₅ | 0 | 2,4-Cl₂ | |
| 1.40 | C₂H₅ | 0 | 2,5-Cl₂ | |
| 1.41 | C₂H₅ | 0 | 2,6-Cl₂ | |
| 1.42 | C₂H₅ | 0 | 3,4-Cl₂ | |
| 1.43 | C₂H₅ | 0 | 3,5-Cl₂ | |
| 1.44 | C₂H₅ | 0 | 3-CF₃ | |
| 1.45 | C₂H₅ | 0 | 4-CF₃ | |
| 1.46 | C₂H₅ | 1 | H | |
| 1.47 | C₂H₅ | 1 | 2-F | |
| 1.48 | C₂H₅ | 1 | 3-F | |
| 1.49 | C₂H₅ | 1 | 4-F | |
| 1.50 | C₂H₅ | 1 | 2-Cl | |
| 1.51 | C₂H₅ | 1 | 3-Cl | |
| 1.52 | C₂H₅ | 1 | 4-Cl | |
| 1.53 | C₂H₅ | 1 | 2,3-Cl₂ | |
| 1.54 | C₂H₅ | 1 | 2,4-Cl₂ | |
| 1.55 | C₂H₅ | 1 | 2,5-Cl₂ | |
| 1.56 | C₂H₅ | 1 | 2,6-Cl₂ | |
| 1.57 | C₂H₅ | 1 | 3,4-Cl₂ | |
| 1.58 | C₂H₅ | 1 | 3,5-Cl₂ | |
| 1.59 | C₂H₅ | 1 | 3-CF₃ | |
| 1.60 | C₂H₅ | 1 | 4-CF₃ | |
| 1.61 | n-C₃H₇ | 0 | H | |
| 1.62 | n-C₃H₇ | 0 | 2-F | |
| 1.63 | n-C₃H₇ | 0 | 3-F | |
| 1.64 | n-C₃H₇ | 0 | 4-F | |
| 1.65 | n-C₃H₇ | 0 | 2-Cl | |
| 1.66 | n-C₃H₇ | 0 | 3-Cl | |
| 1.67 | n-C₃H₇ | 0 | 4-Cl | |
| 1.68 | n-C₃H₇ | 0 | 2,3-Cl₂ | |
| 1.69 | n-C₃H₇ | 0 | 2,4-Cl₂ | |
| 1.70 | n-C₃H₇ | 0 | 2,5-Cl₂ | |
| 1.71 | n-C₃H₇ | 0 | 2,6-Cl₂ | |
| 1.72 | n-C₃H₇ | 0 | 3,4-Cl₂ | |
| 1.73 | n-C₃H₇ | 0 | 3,5-Cl₂ | |
| 1.74 | n-C₃H₇ | 0 | 3-CF₃ | |
| 1.75 | n-C₃H₇ | 0 | 4-CF₃ | |
| 1.76 | n-C₃H₇ | 1 | H | |
| 1.77 | n-C₃H₇ | 1 | 2-F | |
| 1.78 | n-C₃H₇ | 1 | 3-F | |
| 1.79 | n-C₃H₇ | 1 | 4-F | |
| 1.80 | n-C₃H₇ | 1 | 2-Cl | |
| 1.81 | n-C₃H₇ | 1 | 3-Cl | |
| 1.82 | n-C₃H₇ | 1 | 4-Cl | |
| 1.83 | n-C₃H₇ | 1 | 2,3-Cl₂ | |
| 1.84 | n-C₃H₇ | 1 | 2,4-Cl₂ | |
| 1.85 | n-C₃H₇ | 1 | 2,5-Cl₂ | |
| 1.86 | n-C₃H₇ | 1 | 2,6-Cl₂ | |
| 1.87 | n-C₃H₇ | 1 | 3,4-Cl₂ | |
| 1.88 | n-C₃H₇ | 1 | 3,5-Cl₂ | |
| 1.89 | n-C₃H₇ | 1 | 3-CF₃ | |
| 1.90 | n-C₃H₇ | 1 | 4-CF₃ | |
| 1.91 | n-C₄H₉ | 0 | H | |
| 1.92 | n-C₄H₉ | 0 | 2-F | |
| 1.93 | n-C₄H₉ | 0 | 3-F | |
| 1.94 | n-C₄H₉ | 0 | 4-F | |
| 1.95 | n-C₄H₉ | 0 | 2-Cl | |
| 1.96 | n-C₄H₉ | 0 | 3-Cl | |
| 1.97 | n-C₄H₉ | 0 | 4-Cl | |
| 1.98 | n-C₄H₉ | 0 | 2,3-Cl₂ | |
| 1.99 | n-C₄H₉ | 0 | 2,4-Cl₂ | |
| 1.100 | n-C₄H₉ | 0 | 2,5-Cl₂ | |
| 1.101 | n-C₄H₉ | 0 | 2,6-Cl₂ | |
| 1.102 | n-C₄H₉ | 0 | 3,4-Cl₂ | |
| 1.103 | n-C₄H₉ | 0 | 3,5-Cl₂ | |
| 1.104 | n-C₄H₉ | 0 | 3-CF₃ | |
| 1.105 | n-C₄H₉ | 0 | 4-CF₃ | |
| 1.106 | n-C₄H₉ | 1 | H | |
| 1.107 | n-C₄H₉ | 1 | 2-F | |
| 1.108 | n-C₄H₉ | 1 | 3-F | |
| 1.109 | n-C₄H₉ | 1 | 4-F | |
| 1.110 | n-C₄H₉ | 1 | 2-Cl | |
| 1.111 | n-C₄H₉ | 1 | 3-Cl | |
| 1.112 | n-C₄H₉ | 1 | 4-Cl | |
| 1.113 | n-C₄H₉ | 1 | 2,3-Cl₂ | |
| 1.114 | n-C₄H₉ | 1 | 2,4-Cl₂ | |
| 1.115 | n-C₄H₉ | 1 | 2,5-Cl₂ | |
| 1.116 | n-C₄H₉ | 1 | 2,6-Cl₂ | |
| 1.117 | n-C₄H₉ | 1 | 3,4-Cl₂ | |
| 1.118 | n-C₄H₉ | 1 | 3,5-Cl₂ | |
| 1.119 | n-C₄H₉ | 1 | 3-CF₃ | |
| 1.120 | n-C₄H₉ | 1 | 4-CF₃ | |
| 1.121 | CH₂C₆H₅ | 0 | H | |
| 1.122 | CH₂C₆H₅ | 0 | 2-F | |
| 1.123 | CH₂C₆H₅ | 0 | 3-F | |
| 1.124 | CH₂C₆H₅ | 0 | 4-F | |
| 1.125 | CH₂C₆H₅ | 0 | 2-Cl | |
| 1.126 | CH₂C₆H₅ | 0 | 3-Cl | |
| 1.127 | CH₂C₆H₅ | 0 | 4-Cl | |
| 1.128 | CH₂C₆H₅ | 0 | 2,3-Cl₂ | |
| 1.129 | CH₂C₆H₅ | 0 | 2,4-Cl₂ | |
| 1.130 | CH₂C₆H₅ | 0 | 2,5-Cl₂ | |
| 1.131 | CH₂C₆H₅ | 0 | 2,6-Cl₂ | |
| 1.132 | CH₂C₆H₅ | 0 | 3,4-Cl₂ | |
| 1.133 | CH₂C₆H₅ | 0 | 3,5-Cl₂ | |
| 1.134 | CH₂C₆H₅ | 0 | 3-CF₃ | |
| 1.135 | CH₂C₆H₅ | 0 | 4-CF₃ | |
| 1.136 | CH₂C₆H₅ | 1 | H | |
| 1.137 | CH₂C₆H₅ | 1 | 2-F | |
| 1.138 | CH₂C₆H₅ | 1 | 3-F | |
| 1.139 | CH₂C₆H₅ | 1 | 4-F | |
| 1.140 | CH₂C₆H₅ | 1 | 2-Cl | |
| 1.141 | CH₂C₆H₅ | 1 | 3-Cl | |
| 1.142 | CH₂C₆H₅ | 1 | 4-Cl | |
| 1.143 | CH₂C₆H₅ | 1 | 2,3-Cl₂ | |
| 1.144 | CH₂C₆H₅ | 1 | 2,4-Cl₂ | |
| 1.145 | CH₂C₆H₅ | 1 | 2,5-Cl₂ | |
| 1.146 | CH₂C₆H₅ | 1 | 2,6-Cl₂ | |
| 1.147 | CH₂C₆H₅ | 1 | 3,4-Cl₂ | |
| 1.148 | CH₂C₆H₅ | 1 | 3,5-Cl₂ | |
| 1.149 | CH₂C₆H₅ | 1 | 3-CF₃ | |
| 1.150 | CH₂C₆H₅ | 1 | 4-CF₃ | |
| 1.151 | C₆H₅ | 0 | H | |
| 1.152 | C₆H₅ | 0 | 2-F | |
| 1.153 | C₆H₅ | 0 | 3-F | |
| 1.154 | C₆H₅ | 0 | 4-F | |
| 1.155 | C₆H₅ | 0 | 2-Cl | |
| 1.156 | C₆H₅ | 0 | 3-Cl | |
| 1.157 | C₆H₅ | 0 | 4-Cl | |
| 1.158 | C₆H₅ | 0 | 2,3-Cl₂ | |
| 1.159 | C₆H₅ | 0 | 2,4-Cl₂ | |
| 1.160 | C₆H₅ | 0 | 2,5-Cl₂ | |
| 1.161 | C₆H₅ | 0 | 2,6-Cl₂ | |
| 1.162 | C₆H₅ | 0 | 3,4-Cl₂ | |
| 1.163 | C₆H₅ | 0 | 3,5-Cl₂ | |
| 1.164 | C₆H₅ | 0 | 3-CF₃ | |
| 1.165 | C₆H₅ | 0 | 4-CF₃ | |
| 1.166 | C₆H₅ | 1 | H | |
| 1.167 | C₆H₅ | 1 | 2-F | |
| 1.168 | C₆H₅ | 1 | 3-F | |
| 1.169 | C₆H₅ | 1 | 4-F | |
| 1.170 | C₆H₅ | 1 | 2-Cl | |
| 1.171 | C₆H₅ | 1 | 3-Cl | |
| 1.172 | C₆H₅ | 1 | 4-Cl | |
| 1.173 | C₆H₅ | 1 | 2,3-Cl₂ | |
| 1.174 | C₆H₅ | 1 | 2,4-Cl₂ | |
| 1.175 | C₆H₅ | 1 | 2,5-Cl₂ | |
| 1.176 | C₆H₅ | 1 | 2,6-Cl₂ | |
| 1.177 | C₆H₅ | 1 | 3,4-Cl₂ | |
| 1.178 | C₆H₅ | 1 | 3,5-Cl₂ | |
| 1.179 | C₆H₅ | 1 | 3-CF₃ | |
| 1.180 | C₆H₅ | 1 | 4-CF₃ | |
| 1.181 | C₆H₄-4-CH₃ | 0 | H | |
| 1.182 | C₆H₄-4-CH₃ | 0 | 2-F | |
| 1.183 | C₆H₄-4-CH₃ | 0 | 3-F | |
| 1.184 | C₆H₄-4-CH₃ | 0 | 4-F | |
| 1.185 | C₆H₄-4-CH₃ | 0 | 2-Cl | |
| 1.186 | C₆H₄-4-CH₃ | 0 | 3-Cl | |
| 1.187 | C₆H₄-4-CH₃ | 0 | 4-Cl | |
| 1.188 | C₆H₄-4-CH₃ | 0 | 2,3-Cl₂ | |
| 1.189 | C₆H₄-4-CH₃ | 0 | 2,4-Cl₂ | |
| 1.190 | C₆H₄-4-CH₃ | 0 | 2,5-Cl₂ | |
| 1.191 | C₆H₄-4-CH₃ | 0 | 2,6-Cl₂ | |
| 1.192 | C₆H₄-4-CH₃ | 0 | 3,4-Cl₂ | |
| 1.193 | C₆H₄-4-CH₃ | 0 | 3,5-Cl₂ | |
| 1.194 | C₆H₄-4-CH₃ | 0 | 3-CF₃ | |
| 1.195 | C₆H₄-4-CH₃ | 0 | 4-CF₃ | |
| 1.196 | C₆H₄-4-CH₃ | 1 | H | |
| 1.197 | C₆H₄-4-CH₃ | 1 | 2-F | |
| 1.198 | C₆H₄-4-CH₃ | 1 | 3-F | |
| 1.199 | C₆H₄-4-CH₃ | 1 | 4-F | |
| 1.200 | C₆H₄-4-CH₃ | 1 | 2-Cl | |
| 1.201 | C₆H₄-4-CH₃ | 1 | 3-Cl | |
| 1.202 | C₆H₄-4-CH₃ | 1 | 4-Cl | |
| 1.203 | C₆H₄-4-CH₃ | 1 | 2,3-Cl₂ | |
| 1.204 | C₆H₄-4-CH₃ | 1 | 2,4-Cl₂ | |
| 1.205 | C₆H₄-4-CH₃ | 1 | 2,5-Cl₂ | |
| 1.206 | C₆H₄-4-CH₃ | 1 | 2,6-Cl₂ | |
| 1.207 | C₆H₄-4-CH₃ | 1 | 3,4-Cl₂ | |
| 1.208 | C₆H₄-4-CH₃ | 1 | 3,5-Cl₂ | |
| 1.209 | C₆H₄-4-CH₃ | 1 | 3-CF₃ | |
| 1.210 | C₆H₄-4-CH₃ | 1 | 4-CF₃ | |
| 1.211 | C₆H₄-4-F | 0 | H | |
| 1.212 | C₆H₄-4-F | 0 | 2-F | |
| 1.213 | C₆H₄-4-F | 0 | 3-F | |
| 1.214 | C₆H₄-4-F | 0 | 4-F | |
| 1.215 | C₆H₄-4-F | 0 | 2-Cl | |
| 1.216 | C₆H₄-4-F | 0 | 3-Cl | |
| 1.217 | C₆H₄-4-F | 0 | 4-Cl | |
| 1.218 | C₆H₄-4-F | 0 | 2,3-Cl₂ | |
| 1.219 | C₆H₄-4-F | 0 | 2,4-Cl₂ | |
| 1.220 | C₆H₄-4-F | 0 | 2,5-Cl₂ | |
| 1.221 | C₆H₄-4-F | 0 | 2,6-Cl₂ | |
| 1.222 | C₆H₄-4-F | 0 | 3,4-Cl₂ | |
| 1.223 | C₆H₄-4-F | 0 | 3,5-Cl₂ | |
| 1.224 | C₆H₄-4-F | 0 | 3-CF₃ | |
| 1.225 | C₆H₄-4-F | 0 | 4-CF₃ | |
| 1.226 | C₆H₄-4-F | 1 | H | |
| 1.227 | C₆H₄-4-F | 1 | 2-F | |
| 1.228 | C₆H₄-4-F | 1 | 3-F | |
| 1.229 | C₆H₄-4-F | 1 | 4-F | |
| 1.230 | C₆H₄-4-F | 1 | 2-Cl | |
| 1.231 | C₆H₄-4-F | 1 | 3-Cl | |
| 1.232 | C₆H₄-4-F | 1 | 4-Cl | |
| 1.233 | C₆H₄-4-F | 1 | 2,3-Cl₂ | |
| 1.234 | C₆H₄-4-F | 1 | 2,4-Cl₂ | |
| 1.235 | C₆H₄-4-F | 1 | 2,5-Cl₂ | |
| 1.236 | C₆H₄-4-F | 1 | 2,6-Cl₂ | |
| 1.237 | C₆H₄-4-F | 1 | 3,4-Cl₂ | |
| 1.238 | C₆H₄-4-F | 1 | 3,5-Cl₂ | |
| 1.239 | C₆H₄-4-F | 1 | 3-CF₃ | |
| 1.240 | C₆H₄-4-F | 1 | 4-CF₃ | |
| 1.241 | C₆H₄-4-Cl | 0 | H | |
| 1.242 | C₆H₄-4-Cl | 0 | 2-F | |
| 1.243 | C₆H₄-4-Cl | 0 | 3-F | |
| 1.244 | C₆H₄-4-Cl | 0 | 4-F | |
| 1.245 | C₆H₄-4-Cl | 0 | 2-Cl | |
| 1.246 | C₆H₄-4-Cl | 0 | 3-Cl | |
| 1.247 | C₆H₄-4-Cl | 0 | 4-Cl | |
| 1.248 | C₆H₄-4-Cl | 0 | 2,3-Cl₂ | |
| 1.249 | C₆H₄-4-Cl | 0 | 2,4-Cl₂ | |
| 1.250 | C₆H₄-4-Cl | 0 | 2,5-Cl₂ | |
| 1.251 | C₆H₄-4-Cl | 0 | 2,6-Cl₂ | |
| 1.252 | C₆H₄-4-Cl | 0 | 3,4-Cl₂ | |
| 1.253 | C₆H₄-4-Cl | 0 | 3,5-Cl₂ | |
| 1.254 | C₆H₄-4-Cl | 0 | 3-CF₃ | |
| 1.255 | C₆H₄-4-Cl | 0 | 4-CF₃ | |
| 1.256 | C₆H₄-4-Cl | 1 | H | |
| 1.257 | C₆H₄-4-Cl | 1 | 2-F | |
| 1.258 | C₆H₄-4-Cl | 1 | 3-F | |
| 1.259 | C₆H₄-4-Cl | 1 | 4-F | |
| 1.260 | C₆H₄-4-Cl | 1 | 2-Cl | |
| 1.261 | C₆H₄-4-Cl | 1 | 3-Cl | |
| 1.262 | C₆H₄-4-Cl | 1 | 4-Cl | |
| 1.263 | C₆H₄-4-Cl | 1 | 2,3-Cl₂ | |
| 1.264 | C₆H₄-4-Cl | 1 | 2,4-Cl₂ | |
| 1.265 | C₆H₄-4-Cl | 1 | 2,5-Cl₂ | |
| 1.266 | C₆H₄-4-Cl | 1 | 2,6-Cl₂ | |
| 1.267 | C₆H₄-4-Cl | 1 | 3,4-Cl₂ | |
| 1.268 | C₆H₄-4-Cl | 1 | 3,5-Cl₂ | |
| 1.269 | C₆H₄-4-Cl | 1 | 3-CF₃ | |
| 1.270 | C₆H₄-4-Cl | 1 | 4-CF₃ | |
| 1.271 | C₆H₄-4-Br | 0 | H | |
| 1.272 | C₆H₄-4-Br | 0 | 2-F | |
| 1.273 | C₆H₄-4-Br | 0 | 3-F | |
| 1.274 | C₆H₄-4-Br | 0 | 4-F | |
| 1.275 | C₆H₄-4-Br | 0 | 2-Cl | |
| 1.276 | C₆H₄-4-Br | 0 | 3-Cl | |
| 1.277 | C₆H₄-4-Br | 0 | 4-Cl | |
| 1.278 | C₆H₄-4-Br | 0 | 2,3-Cl₂ | |
| 1.279 | C₆H₄-4-Br | 0 | 2,4-Cl₂ | |
| 1.280 | C₆H₄-4-Br | 0 | 2,5-Cl₂ | |
| 1.281 | C₆H₄-4-Br | 0 | 2,6-Cl₂ | |
| 1.282 | C₆H₄-4-Br | 0 | 3,4-Cl₂ | |
| 1.283 | C₆H₄-4-Br | 0 | 3,5-Cl₂ | |
| 1.284 | C₆H₄-4-Br | 0 | 3-CF₃ | |
| 1.285 | C₆H₄-4-Br | 0 | 4-CF₃ | |
| 1.286 | C₆H₄-4-Br | 1 | H | |
| 1.287 | C₆H₄-4-Br | 1 | 2-F | |
| 1.288 | C₆H₄-4-Br | 1 | 3-F | |
| 1.289 | C₆H₄-4-Br | 1 | 4-F | |
| 1.290 | C₆H₄-4-Br | 1 | 2-Cl | |
| 1.291 | C₆H₄-4-Br | 1 | 3-Cl | |
| 1.292 | C₆H₄-4-Br | 1 | 4-Cl | |
| 1.293 | C₆H₄-4-Br | 1 | 2,3-Cl₂ | |
| 1.294 | C₆H₄-4-Br | 1 | 2,4-Cl₂ | |
| 1.295 | C₆H₄-4-Br | 1 | 2,5-Cl₂ | |
| 1.296 | C₆H₄-4-Br | 1 | 2,6-Cl₂ | |
| 1.297 | C₆H₄-4-Br | 1 | 3,4-Cl₂ | |
| 1.298 | C₆H₄-4-Br | 1 | 3,5-Cl₂ | |
| 1.299 | C₆H₄-4-Br | 1 | 3-CF₃ | |
| 1.300 | C₆H₄-4-Br | 1 | 4-CF₃ | |
| 1.301 | C₆H₄-4-OCH₃ | 0 | H | |
| 1.302 | C₆H₄-4-OCH₃ | 0 | 2-F | |
| 1.303 | C₆H₄-4-OCH₃ | 0 | 3-F | |
| 1.304 | C₆H₄-4-OCH₃ | 0 | 4-F | |
| 1.305 | C₆H₄-4-OCH₃ | 0 | 2-Cl | |
| 1.306 | C₆H₄-4-OCH₃ | 0 | 3-Cl | |
| 1.307 | C₆H₄-4-OCH₃ | 0 | 4-Cl | |
| 1.308 | C₆H₄-4-OCH₃ | 0 | 2,3-Cl₂ | |
| 1.309 | C₆H₄-4-OCH₃ | 0 | 2,4-Cl₂ | |
| 1.310 | C₆H₄-4-OCH₃ | 0 | 2,5-Cl₂ | |
| 1.311 | C₆H₄-4-OCH₃ | 0 | 2,6-Cl₂ | |
| 1.312 | C₆H₄-4-OCH₃ | 0 | 3,4-Cl₂ | |
| 1.313 | C₆H₄-4-OCH₃ | 0 | 3,5-Cl₂ | |
| 1.314 | C₆H₄-4-OCH₃ | 0 | 3-CF₃ | |
| 1.315 | C₆H₄-4-OCH₃ | 0 | 4-CF₃ | |
| 1.316 | C₆H₄-4-OCH₃ | 1 | H | |
| 1.317 | C₆H₄-4-OCH₃ | 1 | 2-F | |
| 1.318 | C₆H₄-4-OCH₃ | 1 | 3-F | |
| 1.319 | C₆H₄-4-OCH₃ | 1 | 4-F | |
| 1.320 | C₆H₄-4-OCH₃ | 1 | 2-Cl | |
| 1.321 | C₆H₄-4-OCH₃ | 1 | 3-Cl | |
| 1.322 | C₆H₄-4-OCH₃ | 1 | 4-Cl | |
| 1.323 | C₆H₄-4-OCH₃ | 1 | 2,3-Cl₂ | |
| 1.324 | C₆H₄-4-OCH₃ | 1 | 2,4-Cl₂ | |
| 1.325 | C₆H₄-4-OCH₃ | 1 | 2,5-Cl₂ | |
| 1.326 | C₆H₄-4-OCH₃ | 1 | 2,6-Cl₂ | |
| 1.327 | C₆H₄-4-OCH₃ | 1 | 3,4-Cl₂ | |
| 1.328 | C₆H₄-4-OCH₃ | 1 | 3,5-Cl₂ | |
| 1.329 | C₆H₄-4-OCH₃ | 1 | 3-CF₃ | |
| 1.330 | C₆H₄-4-OCH₃ | 1 | 4-CF₃ | |
| 1.331 | C₆H₄-2-CF₃ | 0 | H | |
| 1.332 | C₆H₄-2-CF₃ | 0 | 2-F | |
| 1.333 | C₆H₄-2-CF₃ | 0 | 3-F | |
| 1.334 | C₆H₄-2-CF₃ | 0 | 4-F | |
| 1.335 | C₆H₄-2-CF₃ | 0 | 2-Cl | |
| 1.336 | C₆H₄-2-CF₃ | 0 | 3-Cl | |
| 1.337 | C₆H₄-2-CF₃ | 0 | 4-Cl | |
| 1.338 | C₆H₄-2-CF₃ | 0 | 2,3-Cl₂ | |
| 1.339 | C₆H₄-2-CF₃ | 0 | 2,4-Cl₂ | |
| 1.340 | C₆H₄-2-CF₃ | 0 | 2,5-Cl₂ | |
| 1.341 | C₆H₄-2-CF₃ | 0 | 2,6-Cl₂ | |
| 1.342 | C₆H₄-2-CF₃ | 0 | 3,4-Cl₂ | |
| 1.343 | C₆H₄-2-CF₃ | 0 | 3,5-Cl₂ | |
| 1.344 | C₆H₄-2-CF₃ | 0 | 3-CF₃ | |
| 1.345 | C₆H₄-2-CF₃ | 0 | 4-CF₃ | |
| 1.346 | C₆H₄-2-CF₃ | 1 | H | |
| 1.347 | C₆H₄-2-CF₃ | 1 | 2-F | |
| 1.348 | C₆H₄-2-CF₃ | 1 | 3-F | |
| 1.349 | C₆H₄-2-CF₃ | 1 | 4-F | |
| 1.350 | C₆H₄-2-CF₃ | 1 | 2-Cl | |
| 1.351 | C₆H₄-2-CF₃ | 1 | 3-Cl | |
| 1.352 | C₆H₄-2-CF₃ | 1 | 4-Cl | |
| 1.353 | C₆H₄-2-CF₃ | 1 | 2,3-Cl₂ | |
| 1.354 | C₆H₄-2-CF₃ | 1 | 2,4-Cl₂ | |
| 1.355 | C₆H₄-2-CF₃ | 1 | 2,5-Cl₂ | |
| 1.356 | C₆H₄-2-CF₃ | 1 | 2,6-Cl₂ | |
| 1.357 | C₆H₄-2-CF₃ | 1 | 3,4-Cl₂ | |
| 1.358 | C₆H₄-2-CF₃ | 1 | 3,5-Cl₂ | |
| 1.359 | C₆H₄-2-CF₃ | 1 | 3-CF₃ | |
| 1.360 | C₆H₄-2-CF₃ | 1 | 4-CF₃ | |
| 1.361 | C₆H₄-3-CF₃ | 0 | H | |
| 1.362 | C₆H₄-3-CF₃ | 0 | 2-F | |
| 1.363 | C₆H₄-3-CF₃ | 0 | 3-F | |
| 1.364 | C₆H₄-3-CF₃ | 0 | 4-F | |
| 1.365 | C₆H₄-3-CF₃ | 0 | 2-Cl | |
| 1.366 | C₆H₄-3-CF₃ | 0 | 3-Cl | |
| 1.367 | C₆H₄-3-CF₃ | 0 | 4-Cl | |
| 1.368 | C₆H₄-3-CF₃ | 0 | 2,3-Cl₂ | |
| 1.369 | C₆H₄-3-CF₃ | 0 | 2,4-Cl₂ | |
| 1.370 | C₆H₄-3-CF₃ | 0 | 2,5-Cl₂ | |
| 1.371 | C₆H₄-3-CF₃ | 0 | 2,6-Cl₂ | |
| 1.372 | C₆H₄-3-CF₃ | 0 | 3,4-Cl₂ | |
| 1.373 | C₆H₄-3-CF₃ | 0 | 3,5-Cl₂ | |
| 1.374 | C₆H₄-3-CF₃ | 0 | 3-CF₃ | |
| 1.375 | C₆H₄-3-CF₃ | 0 | 4-CF₃ | |
| 1.376 | C₆H₄-3-CF₃ | 1 | H | |
| 1.377 | C₆H₄-3-CF₃ | 1 | 2-F | |
| 1.378 | C₆H₄-3-CF₃ | 1 | 3-F | |
| 1.379 | C₆H₄-3-CF₃ | 1 | 4-F | |
| 1.380 | C₆H₄-3-CF₃ | 1 | 2-Cl | |
| 1.381 | C₆H₄-3-CF₃ | 1 | 3-Cl | |
| 1.382 | C₆H₄-3-CF₃ | 1 | 4-Cl | |
| 1.383 | C₆H₄-3-CF₃ | 1 | 2,3-Cl₂ | |
| 1.384 | C₆H₄-3-CF₃ | 1 | 2,4-Cl₂ | |
| 1.385 | C₆H₄-3-CF₃ | 1 | 2,5-Cl₂ | |
| 1.386 | C₆H₄-3-CF₃ | 1 | 2,6-Cl₂ | |
| 1.387 | C₆H₄-3-CF₃ | 1 | 3,4-Cl₂ | |
| 1.388 | C₆H₄-3-CF₃ | 1 | 3,5-Cl₂ | |
| 1.389 | C₆H₄-3-CF₃ | 1 | 3-CF₃ | |
| 1.390 | C₆H₄-3-CF₃ | 1 | 4-CF₃ | |
| 1.391 | C₆H₄-4-CF₃ | 0 | H | |
| 1.392 | C₆H₄-4-CF₃ | 0 | 2-F | |
| 1.393 | C₆H₄-4-CF₃ | 0 | 3-F | |
| 1.394 | C₆H₄-4-CF₃ | 0 | 4-F | |
| 1.395 | C₆H₄-4-CF₃ | 0 | 2-Cl | |
| 1.396 | C₆H₄-4-CF₃ | 0 | 3-Cl | |
| 1.397 | C₆H₄-4-CF₃ | 0 | 4-Cl | |
| 1.398 | C₆H₄-4-CF₃ | 0 | 2,3-Cl₂ | |
| 1.399 | C₆H₄-4-CF₃ | 0 | 2,4-Cl₂ | |
| 1.400 | C₆H₄-4-CF₃ | 0 | 2,5-Cl₂ | |
| 1.401 | C₆H₄-4-CF₃ | 0 | 2,6-Cl₂ | |
| 1.402 | C₆H₄-4-CF₃ | 0 | 3,4-Cl₂ | |
| 1.403 | C₆H₄-4-CF₃ | 0 | 3,5-Cl₂ | |
| 1.404 | C₆H₄-4-CF₃ | 0 | 3-CF₃ | |
| 1.405 | C₆H₄-4-CF₃ | 0 | 4-CF₃ | |
| 1.406 | C₆H₄-4-CF₃ | 1 | H | |
| 1.407 | C₆H₄-4-CF₃ | 1 | 2-F | |
| 1.408 | C₆H₄-4-CF₃ | 1 | 3-F | |
| 1.409 | C₆H₄-4-CF₃ | 1 | 4-F | |
| 1.410 | C₆H₄-4-CF₃ | 1 | 2-Cl | |
| 1.411 | C₆H₄-4-CF₃ | 1 | 3-Cl | |
| 1.412 | C₆H₄-4-CF₃ | 1 | 4-Cl | |
| 1.413 | C₆H₄-4-CF₃ | 1 | 2,3-Cl₂ | |
| 1.414 | C₆H₄-4-CF₃ | 1 | 2,4-Cl₂ | |
| 1.415 | C₆H₄-4-CF₃ | 1 | 2,5-Cl₂ | |
| 1.416 | C₆H₄-4-CF₃ | 1 | 2,6-Cl₂ | |
| 1.417 | C₆H₄-4-CF₃ | 1 | 3,4-Cl₂ | |
| 1.418 | C₆H₄-4-CF₃ | 1 | 3,5-Cl₂ | |
| 1.419 | C₆H₄-4-CF₃ | 1 | 3-CF₃ | |
| 1.420 | C₆H₄-4-CF₃ | 1 | 4-CF₃ | |
| 1.421 | C₆H₄-3-NO₂ | 0 | H | |
| 1.422 | C₆H₄-3-NO₂ | 0 | 2-F | |
| 1.423 | C₆H₄-3-NO₂ | 0 | 3-F | |
| 1.424 | C₆H₄-3-NO₂ | 0 | 4-F | |
| 1.425 | C₆H₄-3-NO₂ | 0 | 2-Cl | |
| 1.426 | C₆H₄-3-NO₂ | 0 | 3-Cl | |
| 1.427 | C₆H₄-3-NO₂ | 0 | 4-Cl | |
| 1.428 | C₆H₄-3-NO₂ | 0 | 2,3-Cl₂ | |
| 1.429 | C₆H₄-3-NO₂ | 0 | 2,4-Cl₂ | |
| 1.430 | C₆H₄-3-NO₂ | 0 | 2,5-Cl₂ | |
| 1.431 | C₆H₄-3-NO₂ | 0 | 2,6-Cl₂ | |
| 1.432 | C₆H₄-3-NO₂ | 0 | 3,4-Cl₂ | |
| 1.433 | C₆H₄-3-NO₂ | 0 | 3,5-Cl₂ | |
| 1.434 | C₆H₄-3-NO₂ | 0 | 3-CF₃ | |
| 1.435 | C₆H₄-3-NO₂ | 0 | 4-CF₃ | |
| 1.436 | C₆H₄-3-NO₂ | 1 | H | |
| 1.437 | C₆H₄-3-NO₂ | 1 | 2-F | |
| 1.438 | C₆H₄-3-NO₂ | 1 | 3-F | |
| 1.439 | C₆H₄-3-NO₂ | 1 | 4-F | |
| 1.440 | C₆H₄-3-NO₂ | 1 | 2-Cl | |
| 1.441 | C₆H₄-3-NO₂ | 1 | 3-Cl | |
| 1.442 | C₆H₄-3-NO₂ | 1 | 4-Cl | |
| 1.443 | C₆H₄-3-NO₂ | 1 | 2,3-Cl₂ | |
| 1.444 | C₆H₄-3-NO₂ | 1 | 2,4-Cl₂ | |
| 1.445 | C₆H₄-3-NO₂ | 1 | 2,5-Cl₂ | |
| 1.446 | C₆H₄-3-NO₂ | 1 | 2,6-Cl₂ | |
| 1.447 | C₆H₄-3-NO₂ | 1 | 3,4-Cl₂ | |
| 1.448 | C₆H₄-3-NO₂ | 1 | 3,5-Cl₂ | |
| 1.449 | C₆H₄-3-NO₂ | 1 | 3-CF₃ | |
| 1.450 | C₆H₄-3-NO₂ | 1 | 4-CF₃ | |
| 1.451 | C₆H₂-2,3,4-Cl₃ | 0 | H | |
| 1.452 | C₆H₂-2,3,4-Cl₃ | 0 | 2-F | |
| 1.453 | C₆H₂-2,3,4-Cl₃ | 0 | 3-F | |
| 1.454 | C₆H₂-2,3,4-Cl₃ | 0 | 4-F | |
| 1.455 | C₆H₂-2,3,4-Cl₃ | 0 | 2-Cl | |
| 1.456 | C₆H₂-2,3,4-Cl₃ | 0 | 3-Cl | |
| 1.457 | C₆H₂-2,3,4-Cl₃ | 0 | 4-Cl | |
| 1.458 | C₆H₂-2,3,4-Cl₃ | 0 | 2,3-Cl₂ | |
| 1.459 | C₆H₂-2,3,4-Cl₃ | 0 | 2,4-Cl₂ | |
| 1.460 | C₆H₂-2,3,4-Cl₃ | 0 | 2,5-Cl₂ | |
| 1.461 | C₆H₂-2,3,4-Cl₃ | 0 | 2,6-Cl₂ | |
| 1.462 | C₆H₂-2,3,4-Cl₃ | 0 | 3,4-Cl₂ | |
| 1.463 | C₆H₂-2,3,4-Cl₃ | 0 | 3,5-Cl₂ | |
| 1.464 | C₆H₂-2,3,4-Cl₃ | 0 | 3-CF₃ | |
| 1.465 | C₆H₂-2,3,4-Cl₃ | 0 | 4-CF₃ | |
| 1.466 | C₆H₂-2,3,4-Cl₃ | 1 | H | |
| 1.467 | C₆H₂-2,3,4-Cl₃ | 1 | 2-F | |
| 1.468 | C₆H₂-2,3,4-Cl₃ | 1 | 3-F | |
| 1.469 | C₆H₂-2,3,4-Cl₃ | 1 | 4-F | |
| 1.470 | C₆H₂-2,3,4-Cl₃ | 1 | 2-Cl | |
| 1.471 | C₆H₂-2,3,4-Cl₃ | 1 | 3-Cl | |
| 1.472 | C₆H₂-2,3,4-Cl₃ | 1 | 4-Cl | |
| 1.473 | C₆H₂-2,3,4-Cl₃ | 1 | 2,3-Cl₂ | |
| 1.474 | C₆H₂-2,3,4-Cl₃ | 1 | 2,4-Cl₂ | |
| 1.475 | C₆H₂-2,3,4-Cl₃ | 1 | 2,5-Cl₂ | |
| 1.476 | C₆H₂-2,3,4-Cl₃ | 1 | 2,6-Cl₂ | |
| 1.477 | C₆H₂-2,3,4-Cl₃ | 1 | 3,4-Cl₂ | |
| 1.478 | C₆H₂-2,3,4-Cl₃ | 1 | 3,5-Cl₂ | |
| 1.479 | C₆H₂-2,3,4-Cl₃ | 1 | 3-CF₃ | |
| 1.480 | C₆H₂-2,3,4-Cl₃ | 1 | 4-CF₃ | |
| 1.481 | C₆H₂-2,4,5-Cl₃ | 0 | H | |
| 1.482 | C₆H₂-2,4,5-Cl₃ | 0 | 2-F | |
| 1.483 | C₆H₂-2,4,5-Cl₃ | 0 | 3-F | |
| 1.484 | C₆H₂-2,4,5-Cl₃ | 0 | 4-F | |
| 1.485 | C₆H₂-2,4,5-Cl₃ | 0 | 2-Cl | |
| 1.486 | C₆H₂-2,4,5-Cl₃ | 0 | 3-Cl | |
| 1.487 | C₆H₂-2,4,5-Cl₃ | 0 | 4-Cl | |
| 1.488 | C₆H₂-2,4,5-Cl₃ | 0 | 2,3-Cl₂ | |
| 1.489 | C₆H₂-2,4,5-Cl₃ | 0 | 2,4-Cl₂ | |
| 1.490 | C₆H₂-2,4,5-Cl₃ | 0 | 2,5-Cl₂ | |
| 1.491 | C₆H₂-2,4,5-Cl₃ | 0 | 2,6-Cl₂ | |
| 1.492 | C₆H₂-2,4,5-Cl₃ | 0 | 3,4-Cl₂ | |
| 1.493 | C₆H₂-2,4,5-Cl₃ | 0 | 3,5-Cl₂ | |
| 1.494 | C₆H₂-2,4,5-Cl₃ | 0 | 3-CF₃ | |
| 1.495 | C₆H₂-2,4,5-Cl₃ | 0 | 4-CF₃ | |
| 1.496 | C₆H₂-2,4,5-Cl₃ | 1 | H | |
| 1.497 | C₆H₂-2,4,5-Cl₃ | 1 | 2-F | |
| 1.498 | C₆H₂-2,4,5-Cl₃ | 1 | 3-F | |
| 1.499 | C₆H₂-2,4,5-Cl₃ | 1 | 4-F | |
| 1.500 | C₆H₂-2,4,5-Cl₃ | 1 | 2-Cl | |
| 1.501 | C₆H₂-2,4,5-Cl₃ | 1 | 3-Cl | |
| 1.502 | C₆H₂-2,4,5-Cl₃ | 1 | 4-Cl | |
| 1.503 | C₆H₂-2,4,5-Cl₃ | 1 | 2,3-Cl₂ | |
| 1.504 | C₆H₂-2,4,5-Cl₃ | 1 | 2,4-Cl₂ | |
| 1.505 | C₆H₂-2,4,5-Cl₃ | 1 | 2,5-Cl₂ | |
| 1.506 | C₆H₂-2,4,5-Cl₃ | 1 | 2,6-Cl₂ | |
| 1.507 | C₆H₂-2,4,5-Cl₃ | 1 | 3,4-Cl₂ | |
| 1.508 | C₆H₂-2,4,5-Cl₃ | 1 | 3,5-Cl₂ | |
| 1.509 | C₆H₂-2,4,5-Cl₃ | 1 | 3-CF₃ | |
| 1.510 | C₆H₂-2,4,5-Cl₃ | 1 | 4-CF₃ | |
| 1.511 | C₆H₃-2-OCH₃-5-Br | 0 | H | |
| 1.512 | C₆H₃-2-OCH₃-5-Br | 0 | 2-F | |
| 1.513 | C₆H₃-2-OCH₃-5-Br | 0 | 3-F | |
| 1.514 | C₆H₃-2-OCH₃-5-Br | 0 | 4-F | |
| 1.515 | C₆H₃-2-OCH₃-5-Br | 0 | 2-Cl | |
| 1.516 | C₆H₃-2-OCH₃-5-Br | 0 | 3-Cl | |
| 1.517 | C₆H₃-2-OCH₃-5-Br | 0 | 4-Cl | |
| 1.518 | C₆H₃-2-OCH₃-5-Br | 0 | 2,3-Cl₂ | |
| 1.519 | C₆H₃-2-OCH₃-5-Br | 0 | 2,4-Cl₂ | |
| 1.520 | C₆H₃-2-OCH₃-5-Br | 0 | 2,5-Cl₂ | |
| 1.521 | C₆H₃-2-OCH₃-5-Br | 0 | 2,6-Cl₂ | |
| 1.522 | C₆H₃-2-OCH₃-5-Br | 0 | 3,4-Cl₂ | |
| 1.523 | C₆H₃-2-OCH₃-5-Br | 0 | 3,5-Cl₂ | |
| 1.524 | C₆H₃-2-OCH₃-5-Br | 0 | 3-CF₃ | |
| 1.525 | C₆H₃-2-OCH₃-5-Br | 0 | 4-CF₃ | |
| 1.526 | C₆H₃-2-OCH₃-5-Br | 1 | H | |
| 1.527 | C₆H₃-2-OCH₃-5-Br | 1 | 2-F | |
| 1.528 | C₆H₃-2-OCH₃-5-Br | 1 | 3-F | |
| 1.529 | C₆H₃-2-OCH₃-5-Br | 1 | 4-F | |
| 1.530 | C₆H₃-2-OCH₃-5-Br | 1 | 2-Cl | |
| 1.531 | C₆H₃-2-OCH₃-5-Br | 1 | 3-Cl | |
| 1.532 | C₆H₃-2-OCH₃-5-Br | 1 | 4-Cl | |
| 1.533 | C₆H₃-2-OCH₃-5-Br | 1 | 2,3-Cl₂ | |
| 1.534 | C₆H₃-2-OCH₃-5-Br | 1 | 2,4-Cl₂ | |
| 1.535 | C₆H₃-2-OCH₃-5-Br | 1 | 2,5-Cl₂ | |
| 1.536 | C₆H₃-2-OCH₃-5-Br | 1 | 2,6-Cl₂ | |
| 1.537 | C₆H₃-2-OCH₃-5-Br | 1 | 3,4-Cl₂ | |
| 1.538 | C₆H₃-2-OCH₃-5-Br | 1 | 3,5-Cl₂ | |
| 1.539 | C₆H₃-2-OCH₃-5-Br | 1 | 3-CF₃ | |
| 1.540 | C₆H₃-2-OCH₃-5-Br | 1 | 4-CF₃ | |
| 1.541 | 1-naphthyl | 0 | H | |
| 1.542 | 1-naphthyl | 0 | 2-F | |
| 1.543 | 1-naphthyl | 0 | 3-F | |
| 1.544 | 1-naphthyl | 0 | 4-F | |
| 1.545 | 1-naphthyl | 0 | 2-Cl | |
| 1.546 | 1-naphthyl | 0 | 3-Cl | |
| 1.547 | 1-naphthyl | 0 | 4-Cl | |
| 1.548 | 1-naphthyl | 0 | 2,3-Cl₂ | |
| 1.549 | 1-naphthyl | 0 | 2,4-Cl₂ | |
| 1.550 | 1-naphthyl | 0 | 2,5-Cl₂ | |
| 1.551 | 1-naphthyl | 0 | 2,6-Cl₂ | |
| 1.552 | 1-naphthyl | 0 | 3,4-Cl₂ | |
| 1.553 | 1-naphthyl | 0 | 3,5-Cl₂ | |
| 1.554 | 1-naphthyl | 0 | 3-CF₃ | |
| 1.555 | 1-naphthyl | 0 | 4-CF₃ | |
| 1.556 | 1-naphthyl | 1 | H | |
| 1.557 | 1-naphthyl | 1 | 2-F | |
| 1.558 | 1-naphthyl | 1 | 3-F | |
| 1.559 | 1-naphthyl | 1 | 4-F | |
| 1.560 | 1-naphthyl | 1 | 2-Cl | |
| 1.561 | 1-naphthyl | 1 | 3-Cl | |
| 1.562 | 1-naphthyl | 1 | 4-Cl | |
| 1.563 | 1-naphthyl | 1 | 2,3-Cl₂ | |
| 1.564 | 1-naphthyl | 1 | 2,4-Cl₂ | |
| 1.565 | 1-naphthyl | 1 | 2,5-Cl₂ | |
| 1.566 | 1-naphthyl | 1 | 2,6-Cl₂ | |
| 1.567 | 1-naphthyl | 1 | 3,4-Cl₂ | |
| 1.568 | 1-naphthyl | 1 | 3,5-Cl₂ | |
| 1.569 | 1-naphthyl | 1 | 3-CF₃ | |
| 1.570 | 1-naphthyl | 1 | 4-CF₃ | |

### BIOLOGICAL EXAMPLES

A. Control of Animal Parasites

### Example B.1

### In-Vivo Test Against Trichostrongylus colubriformis and Haemonchus contortus in Mongolian Gerbils (Meriones unguiculatus) on Peroral Administration

Six to eight week-old Mongolian gerbils are infected by artificial feeding with about 2000 larvae each of the 3rd stage of T. colubriformis and H. contortus. 6 days after infection, the gerbils are lightly anaesthetized with N₂O and treated by peroral administration with the test compounds, dissolved in a mixture of 2 parts of DMSO and 1 part of polyethylene glycol (PEG 300), using amounts of 100, 32 and 10-0.1 mg/kg. On day 9 (3 days after treatment), when most of the H. contortus larvae of the late 4th stage and most of the T. colubriformis are immature adults, the gerbils are killed to count the worms. The efficacy is calculated in % reduction of the number of worms in each gerbil by comparison with the geometric mean of the number of worms of 8 infected and untreated gerbils.

A strong reduction of the nematode attack is achieved in this test with compounds of the formula (I).

B. Control of Plant Pests

### Example B.2

### Action against Heliothis virescens Caterpillars

Young soybean plants are sprayed with an aqueous emulsion spray mixture comprising 400 ppm of active ingredient and, after the spray-coating has dried, are populated with 10 caterpillars of Heliothis virescens in the first stage and then placed in a plastics container.

Evaluation is carried out 6 days later. The percentage reduction in population and in feeding damage (% activity) are determined by comparing the number of dead caterpillars and the feeding damage on the treated plants with that on untreated plants. The compounds of the Tables exhibit good activity against Heliothis virescens in this test.

### Example B.3

### Action against Plutella xylostella Caterpillars

Young cabbage plants are sprayed with an aqueous emulsion spray mixture comprising 400 ppm of active ingredient and, after the spray-coating has dried, are populated with 10 caterpillars of Plutella xylostella in the third stage and then placed in a plastics container. Evaluation is carried out 3 days later. The percentage reduction In population and in feeding damage (% activity) are determined by comparing the number of dead caterpillars and the feeding damage on the treated plants with that on untreated plants.

The compounds of the Tables exhibit good activity against Plutella xylostella.

### Example B.4

### Action Against Diabrotica balteata Larvae

Maize seedlings are sprayed with an aqueous emulsion spray mixture comprising 400 ppm of active ingredient and, after the spray-coating has dried, are populated with 10 larvae of Diabrotica balteata in the second stage and then placed in a plastics container. Evaluation is carried out 6 days later. The percentage reduction in population (% activity) is determined by comparing the number of dead larvae on the treated plants with that on untreated plants. The compounds of the Tables exhibit good activity against Diabrotica balteata in this test.

1. Compound of the formula (I) 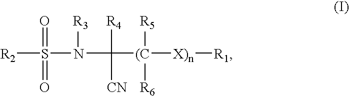
in which
R₁ is aryl or heteroaryl, in each case unsubstituted or mono- or polysubstituted by R₇, where the substituents can in each case be identical or different if their number is greater than 1;
R₂ is C₁-C₆alkyl, halo-C₁-C₆alkyl, C₃-C₈cycloalkyl, halo-C₃-C₈cycloalkyl, NHR₈, aryl or heteroaryl, in each case unsubstituted or mono- or polysubstituted by R₇, where the substituents can in each case be identical or different if their number is greater than 1, or pyrrolidinyl, piperidinyl, imidazolidinyl, piperazinyl, pyrazolidinyl, morpholinyl, indolinyl or isoindolinyl, in each case bonded via N;
R₃ is hydrogen, C₁-C₆alkyl, halo-C₁-C₆alkyl, C₁-C₆alkoxy-C₁-C₆alkyl, benzyl, C₁-C₆alkylheteroaryl, C₁-C₆alkoxycarbonyl or C₁-C₆alkylcarbonyl;
R₄, R₅ and R₆ either independently of one another are hydrogen, halogen, C₁-C₆alkyl, halo-C₁-C₆alkyl, C₁-C₆alkoxy, halo-C₁-C₆alkoxy, C₁-C₆alkylthio, halo-C₁-C₆alkylthio, C₂-C₆alkenyl, C₂-C₆alkynyl, unsubstituted or substituted C₃-C₈cycloalkyl, where the substituents are selected from the group consisting of halogen and C₁-C₆alkyl, or unsubstituted or substituted phenyl, where the substituents are selected from the group consisting of halogen, C₁-C₆alkyl and phenyl;
or R₄ and R₅, together with the carbon atoms to which they are bonded, are a five- to seven-membered, saturated or partially unsaturated heterocyclic ring having 1 to 2 heteroatoms from the group consisting of nitrogen, oxygen and sulphur;
R₇ is halogen, C₁-C₆alkyl, halo-C₁-C₆alkyl, C₁-C₆alkoxy, halo-C₁-C₆alkoxy, C₁-C₆alkylthio, halo-C₁-C₆alkylthio, C₂-C₆alkenyl, C₂-C₆alkynyl; aryl, phenylacetylenyl or heteroaryl, in each case unsubstituted or mono- or polysubstituted, where the substituents are in each case selected from the group consisting of halogen, nitro, cyano, C₁-C₆alkyl, halo-C₁-C₆alkyl, C₁-C₆alkoxy, halo-C₁-C₆alkoxy, and can in each case be identical or different if their number is greater than 1;
R₈ is aryl which is unsubstituted or mono- to pentasubstituted, where the substituents are selected from the group consisting of halogen, nitro, cyano, C₁-C₆alkyl, halo-C₁-C₆alkyl, C₁-C₆alkoxy and halo-C₁-C₆alkoxy, and can be identical or different if their number is greater than 1;
X is O, S, S(O) or S(O)₂; and
n is 1; and, where appropriate, E/Z isomers, mixtures of E/Z isomers and/or tautomers thereof, in each case in free form or in salt form.

2. Compound of the formula (I) according to claim 1, wherein R₁ is aryl which is unsubstituted or mono- to penta-substituted by R₇, where the substitutents in each case can be identical or different if their number is greater than 1.

3. Compound of the formula (I) according to claim 1, wherein R₁ is aryl which is mono- to trisubstituted by R₇, where the substituents in each case can be identical or different if their number is greater than 1.

4. Compound of the formula (I) according to claim 1, wherein R₂ is C₁-C₆alkyl, halo-C₁-C₆alkyl, aryl or heteroaryl which is in each case unsubstituted or mono- to polysubstituted by R₇, where the substituents can in each case be identical or different if their number is greater than 1.

5. Compound of the formula (I) according to claim 1, wherein R₂ is C₁-C₆alkyl, halo-C₁-C₆alkyl or aryl which is unsubstituted or mono- to pentasubstituted by R₇, where the substituents can be identical or different if their number is greater than 1.

6. Compound of the formula (I) according to claim 1, wherein R₂ is aryl which is unsubstituted or mono- to trisubstituted by R₇, where the substituents can be identical or different if their number is greater than 1.

7. Compound of the formula (I) according to claim 1, wherein R₃ is hydrogen or C₁-C₆alkyl.

8. Compound of the formula (I) according to claim 1, wherein R₃ is hydrogen or C₁-C₄alkyl.

9. Compound of the formula (I) according to claim 1, wherein R₃ is hydrogen.

10. Compound of the formula (I) according to claim 1, wherein R₄, R₅ and R₆ independently of one another are hydrogen, C₁-C₆alkyl, halo-C₁-C₆alkyl, C₁-C₆alkoxy, halo-C₁-C₆alkoxy, C₂-C₆alkenyl, C₂-C₆-alkynyl, C₃-C₆cycloalkyl.

11. Compound of the formula (I) according to claim 1, wherein R₄, R₅ and R₆ independently of one another are hydrogen, C₁-C₄alkyl, halo-C₁-C₄alkyl or C₃-C₆cycloalkyl.

12. Compound of the formula (I) according to claim 1, wherein R₄, R₅ and R₆ independently of one another are hydrogen or C₁-C₂alkyl.

13. Compound of the formula (I) according to claim 1, wherein R₇ is halogen, C₁-C₄alkyl, halo-C₁-C₄alkyl, C₁-C₄-alkoxy, halo-C₁-C₄alkoxy; aryl or phenylacetylenyl, in each case unsubstituted or mono- or polysubstituted, where the substituents are selected from the group consisting of halogen, C₁-C₆alkyl, halo-C₁-C₆alkyl, C₁-C₆alkoxy, halo-C₁-C₆alkoxy, and can in each case be identical or different if their number is greater than 1.

14. Compound of the formula (I) according to claim 1, wherein R₇ is halogen, C₁-C₂alkyl, halo-C₁-C₂alkyl, C₁-C₂alkoxy, halo-C₁-C₂alkoxy.

15. Compound of the formula (I) according to claim 1, wherein R₇ is halogen or halo-C₁-C₂alkyl.

16. Compound of the formula (I) according to claim 1, wherein R₈ is unsubstituted or mono- to trisubstituted aryl, where the substituents are selected from the group consisting of halogen, C₁-C₄alkyl, halo-C₁-C₄alkyl, C₁-C₄alkoxy and halo-C₁-C₄alkoxy, and can be identical or different if their number is greater than 1.

17. Compound of the formula (I) according to claim 1, wherein R₈ is mono- to trisubstituted aryl, where the substituents are selected from the group consisting of halogen, C₁-C₂alkyl, halo-C₁-C₂alkyl, and halo-C₁-C₂alkoxy, and can be identical or different if their number is greater than 1.

18. Compound of the formula (I) according to claim 1, wherein R₈ is mono- or disubstituted aryl, where the substituents are selected from the group consisting of halogen and halo-C₁-C₂alkyl, and can be identical or different if their number is greater than 1.

19. Compound of the formula (I) according to claim 1, wherein X is O or S.

20. Compound of the formula (I) according to claim 1, wherein X is O.

21. (canceled)

22. Compound of the formula (I) according to claim 1, wherein R₁ is aryl which is unsubstituted or mono- or pentasubstituted by R₇, where the substituents can in each case be identical or different if their number is greater than 1; R₂ is C₁-C₆alkyl, halo-C₁-C₆alkyl, aryl or heteroaryl, in each case unsubstituted or mono- or polysubstituted by R₇, where the substituents can in each case be identical or different if their number is greater than 1; R₃ is hydrogen or C₁-C₆alkyl; R₄, R₅ and R₆ independently of one another are hydrogen, C₁-C₆alkyl, halo-C₁-C₆alkyl, C₁-C₆alkoxy, halo-C₁-C₆alkoxy, C₂-C₆alkenyl, C₂-C₆alkynyl, C₃-C₆cycloalkyl; R₇ is halogen, C₁-C₄alkyl, halo-C₁-C₄alkyl, C₁-C₄alkoxy, halo-C₁-C₄alkoxy, aryl or phenylacetylenyl, in each case unsubstituted or mono- or polysubstituted, where the substituents are selected from the group consisting of halogen, C₁-C₆alkyl, halo-C₁-C₆alkyl, C₁-C₆alkoxy, halo-C₁-C₆alkoxy, and in each case can be identical or different if their number is greater than 1; R₈ is unsubstituted or mono- to trisubstituted aryl, where the substituents are selected from the group consisting of halogen, C₁-C₄alkyl, halo-C₁-C₄alkyl, C₁-C₄alkoxy and halo-C₁-C₄alkoxy, and can be identical or different if their number is greater than 1; and X is O or S.

23. Compound of the formula (I) according to claim 1, wherein R₁ is aryl which is mono- or trisubstituted by R₇, where the substituents can in each case be identical or different if their number is greater than 1; R₂ is C₁-C₆alkyl, halo-C₁-C₆alkyl or aryl which is unsubstituted or mono- to pentasubstituted by R₇, where the substituents can be identical or different if their number is greater than 1; R₃ is hydrogen or C₁-C₄alkyl; R₄, R₅ and R₆ independently of one another are hydrogen, C₁-C₄alkyl, halo-C₁-C₄alkyl or C₃-C₆cycloalkyl; R₇ is halogen, C₁-C₂alkyl, halo-C₁-C₂alkyl, C₁-C₂alkoxy or halo-C₁-C₂alkoxy; R₈ is mono- to trisubstituted aryl, where the substituents are selected from the group consisting of halogen, C₁-C₂alkyl, halo-C₁-C₂alkyl, and halo-C₁-C₂alkoxy, and can be identical or different if their number is greater than 1; and X is O.

24. Compound of the formula (I) according to claim 1, wherein R₁ is aryl which is mono- to trisubstituted by R₇, where the substituents can in each case be identical or different if their number is greater than 1; R₂ is aryl which is unsubstituted or mono- to trisubstituted by R₇, where the substituents can in each case be identical or different if their number is greater than 1; R₃ is hydrogen; R₄, R₅ and R₆ independently of one another are hydrogen or C₁-C₂alkyl; R₇ is halogen or halo-C₁-C₂alkyl; R₈ is mono- or disubstituted aryl, where the substituents are selected from the group consisting of halogen and halo-C₁-C₂alkyl, and can be identical or different if their number is greater than 1; and X is O.

25. Compound of the formula (I) according to claim 1, named N-(1-cyano-1-[2,3-dichlorophenoxymethyl]ethyl)-C-phenylmethanesulphonamide.

26. Process for the preparation of compounds of the formula (I), in each case in free form or in salt form, according to claim 1, characterized in that a compound of the formula (II) 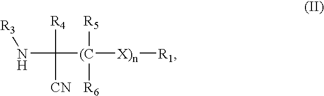
which is known or can be prepared in analogy to corresponding known compounds and in which R₁, R₃, R₄, R₅, R₆, X and n are as defined for the formula (I), is reacted with a compound of the formula (III) 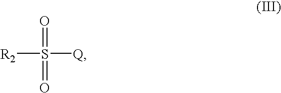
which is known or can be prepared in analogy to corresponding known compounds and in which R₂ are as defined for the formula (I) and Q is a leaving group, if appropriate in the presence of a basic catalyst, and in each case, if desired, a compound of the formula (I), in each case in free form or in salt form, obtainable according to the process or in another manner, is converted into another compound of the formula (I), a mixture of isomers obtainable according to the process is separated and the desired isomer is isolated and/or a free compound of the formula (I) obtainable according to the process is converted into a salt or a salt of a compound of the formula (I) obtainable according to the process is converted into the free compound of the formula (I) or into another salt.

27. Composition for the control of parasites, which, in addition to carriers and/or dispersants, contains as active compound at least one compound of the formula (I) according to claim 1.

28-31. (canceled)

32. A method for controlling parasites comprising applying to said parasites or its habitat a parasiticidal effective amount of at least one compound of formula I of claim 1.

33. The method of claim 33 wherein said parasiticidal effective amount of said at least one compound of formula I of claim 1 is administered to an animal host of said parasite.

34. The method of claim 33 whereby said at least one compound of formula I of claim 1 is administered to said animal host topically, perorally, parenterally, or subcutaneously.

35. The method of claim 32 whereby said compound is in a formulation consisting of the group of pour-on, spot-on, tablet, chewie, powder, boli, capsules, suspension, emulsion, solution, injectable, water-additive, and food-additive.

36. The method of claim 32 wherein said parasites are endo-parasites.

37. The method of claim 36 wherein said endo-parasites are helminthes.

38. A method of treating an animal for parasites comprising administering to said animal in need of treatment thereof a parasiticidal effective amount of the composition of claim 27.

39. The method of claim 38 wherein said administration to said animal is topically, perorally, parenterally, or subcutaneously.

40. The method of claim 38 wherein said composition of claim 27 is in a formulation consisting of the group of pour-on, spot-on, tablet, chewie, powder, boli, capsules, suspension, emulsion, solution, injectable, water-additive, and food-additive.

41. The method of claim 38 wherein said parasites are endo-parasites.

42. The method of claim 41 wherein said endo-parasites are helminthes.
